(19) United States
(12) Patent Application Publication
(10) Pub. No.: US 2026/0234727 A1
(43) Pub. Date: Aug. 13, 2026

(54) PLASMA CFDNA METHYLATION FOR NONINVASIVE MULTIPLE SCLEROSIS DIAGNOSIS, SUBTYPE CLASSIFICATION, AND PROGNOSIS

(71) Applicants: Northwestern University, Evanston, IL (US); University of Pittsburgh, Pittsburgh, PA (US)

(72) Inventors: Yaping LIU, Evanston, IL (US); Zongqi XIA, Pittsburgh, PA (US)

(21) Appl. No.: 19/538,325

(22) Filed: Feb. 12, 2026

Related U.S. Application Data
(60) Provisional application No. 63/757,556, filed on Feb. 12, 2025.

(51) Int. Cl. C12Q 1/6883

(52) U.S. Cl. CPC C12Q 1/6883; C12Q 2600/154

(57) ABSTRACT

Provided herein are circulating cell-free DNA methylation profiles and methods of use thereof as noninvasive multiple sclerosis biomarkers.

### CROSS-REFERENCE

This application claims the benefit of U.S. Provisional Patent Application No. 63/757,556, filed Feb. 12, 2025, and is incorporated by reference herein in its entirety.

### SUPPLEMENTARY TABLES

The specification includes Large Tables. Supplemental Tables 1-11 have been submitted via EFS-Web in electronic format as follows: File name: Supplementary_table_1.txt, Date created: Feb. 12, 2026, File size: 4,096 Bytes; Supplementary_table_2.txt, Date created: Feb. 12, 2026, File size: 8,192 Bytes; Supplementary_table_3.txt, Date created: Feb. 12, 2026, File size: 315,392 Bytes; Supplementary_table_4.txt, Date created: Feb. 12, 2026, File size: 45,056 Bytes; Supplementary_table_5.txt, Date created: Feb. 12, 2026, File size: 32,768 Bytes; Supplementary_table_6.txt, Date created: Feb. 12, 2026, File size: 16,384 Bytes; Supplementary_table_7.txt, Date created: Feb. 12, 2026, File size: 102,400 Bytes; Supplementary_table_8.txt, Date created: Feb. 12, 2026, File size: 16,384 Bytes; Supplementary_table_9.txt, Date created: Feb. 12, 2026, File size: 4,096 Bytes; Supplementary_table_10.txt, Date created: Feb. 12, 2026, File size: 20,480 Bytes; Supplementary_table_11.txt, Date created: Feb. 12, 2026, File size: 581,632 Bytes. The contents of each table are hereby incorporated by reference in their entirety.

LENGTHY TABLES
The patent application contains a lengthy table section. A copy of the table is available in electronic form from the USPTO web site (https://seqdata.uspto.gov/docdetail?docId=US20260234727A1). An electronic copy of the table will also be available from the USPTO upon request and payment of the fee set forth in 37 CFR 1.19(b)(3).

Supplementary Table 1. Participant-level demographic and clinical profile.

Supplementary Table 2. Summary statistics of cfDNA WGBS.

Supplementary Table 3. Differential CpG sites in cfDNA WGBS comparing MS and controls and comparing across MS subtypes.

Supplementary Table 4. Differentially methylated regions in cfDNA WGBS comparing MS and controls and comparing across MS subtypes.

Supplementary Table 5. Gene ontology and motif enrichment at differentially methylated regions in cfDNA WGBS comparing MS and controls and comparing across MS subtypes (from g: profile).

Supplementary Table 6. Tissue-of-origin inferences from cfDNA WGBS in each sample.

Supplementary Table 7. Differential CpG sites in cfDNA WGBS comparing MS patients with higher vs lower patient-reported disability (PDDS≥4 vs <4).

Supplementary Table 8. Differentially methylated regions in cfDNA WGBS comparing MS patients with higher vs lower patient-reported disability severity (PDDS≥4 vs <4).

Supplementary Table 9. Gene ontology and motif enrichment at DMRs in cfDNA WGBS comparing MS patients with higher vs lower patient-reported disability severity (PDDS≥4 vs <4) from g: profile.

Supplementary Table 10. Post-baseline (i.e., sample collection) longitudinal patient-reported disability scores for each participant at different time points.

Supplementary Table 11. Prognostic genomic regions where baseline cfDNA methylation levels were associated with differential patient-reported disability progression.

### FIELD

Provided herein are circulating cell-free DNA methylation profiles and methods of use thereof as noninvasive multiple sclerosis biomarkers.

### BACKGROUND

Multiple sclerosis (MS) is a chronic autoimmune disease impairing the central nervous system (CNS), causing inflammatory demyelination and progressive neurodegeneration in approximately three million individuals globally. Most individuals develop initially Relapsing-Remitting MS (RRMS), defined by discrete episodes of acute neurological symptoms (i.e., relapse) followed by partial or full recovery (i.e., remission), and subsequently transition to Secondary Progressive MS (SPMS) after decades, while a subpopulation develop Primary Progressive MS (PPMS) from the onset. Patients with progressive MS (PMS), including both PPMS and SPMS, experience worsening neurological impairment without discrete periods of relapse or remission. Among the approved disease-modifying therapies (DMTs), all have indications for RRMS or “active” SPMS (i.e., with ongoing relapses). No DMT is yet approved for non-active SPMS (i.e., without relapse), and only one current DMT has indication for PPMS. Early initiation of suitable DMTs improves long-term outcomes, whereas inappropriate DMT selection may cause suboptimal outcomes. Timely MS subtype classification could improve DMT selection, but MS subtyping is retrospective in current practice, potentially delaying appropriate therapeutic guidance. Further, given patient variations in disability progression and treatment response, effective monitoring of disease severity and iterative prognosis could improve clinical care. Magnetic resonance imaging (MRI) is the chief monitoring method in current practice, but has prediction limitations and practical constraints due to access difficulties, patient discomfort, high cost, and insurance restriction.

Non-invasive blood biomarkers provide accessible and cost-effective alternatives for MS subtype classification, disability monitoring, and disease prognosis. Even as biomarkers like neurofilament light chain (NfL) and glial fibrillary acidic protein (GFAP) enter clinical practice, there is room for improvement throughout the clinical contexts of MS care. Although elevated NfL and GFAP levels indicate neuronal and astrocytic injury, respectively, they do not fully capture the multifaceted pathology underlying inflammatory disease activity, progression from RRMS to SPMS, or the steady decline in PMS.

### SUMMARY

Provided herein are circulating cell-free DNA methylation profiles and methods of use thereof as noninvasive multiple sclerosis biomarkers.

In multiple sclerosis (MS), there is a critical need for cost-effective blood biomarkers to concurrently classify disease subtypes, monitor disability severity, and predict long-term progression. Low-coverage whole-genome bisulfite sequencing (WGBS) was performed on 75 plasma cell-free DNA (cfDNA) samples collected from a prospective clinic cohort with longitudinal disability outcomes. cfDNA methylation profile was used for differentiating MS patients from controls, classifying MS subtypes, estimating disability severity, and predicting disease trajectories. Hundreds of differentially methylated regions (DMRs) were identified that significantly distinguished MS from controls, separated MS subtypes, and stratified disability severity levels. These DMRs were highly enriched in immunologically and neurologically relevant cis-regulatory elements and in motifs associated with neuronal function and T-cell differentiation. To distinguish MS subtypes and disability severity, models using DMRs achieved area-under-the-curve (AUC) values ranging of 0.67-0.81 and models using inferred tissue-of-origin patterns from cfDNA methylation achieved AUC 0.70-0.82, outperforming historical benchmark neurofilament light chain (NfL) and glial fibrillary acidic protein (GFAP) in the same cohort. Finally, a linear mixed-effects model identified “prognostic regions” where baseline cfDNA methylation levels were associated with subsequent disability progression and predicted the future disability severity (AUC=0.74) within a 3-year evaluation window. Using these prognostic regions, a cfDNA methylation-based progression risk score and stratified patient groups with differential progression risks were generated. The present findings highlight circulating cfDNA methylation profiles as promising clinical MS biomarkers for future validation using the higher-depth WGBS data.

In some embodiments, provided herein are methods comprising detecting the methylation status of a plurality of biomarkers in a sample comprising cell free DNA (cfDNA) from a subject, wherein each of the plurality of biomarkers is a methylation locus comprising at least a single CpG site and/or differentially methylated region (DMR) that is indicative of multiple sclerosis (MS) status. In some embodiments, the methylation status of one or more (e.g., 1, 2, 5, 10, 20, 50, 100, 200, 500, 1000, or more) of the plurality of biomarkers is indicative of a subject suffering from MS versus not suffering from MS. In some embodiments, the methylation status of one or more (e.g., 1, 2, 5, 10, 20, 50, 100, 200, 500, 1000, or more) of the plurality of biomarkers is indicative of a subject suffering from progressive MS (PMS) versus suffering from relapsing-remitting MS (RRMS). In some embodiments, the methylation status of one or more (e.g., 1, 2, 5, 10, 20, 50, 100, 200, 500, 1000, or more) of the plurality of biomarkers is indicative of a subject suffering from RRMS versus suffering from PMS. In some embodiments, the methylation status of one or more (e.g., 1, 2, 5, 10, 20, 50, 100, 200, 500, 1000, or more) of the plurality of biomarkers is indicative of a subject suffering from primary progressive MS (PPMS) versus suffering from secondary progressive MS (SPMS). In some embodiments, the methylation status of one or more (e.g., 1, 2, 5, 10, 20, 50, 100, 200, 500, 1000, or more) of the plurality of biomarkers is indicative of a subject suffering from SPMS versus suffering from PPMS. In some embodiments, the methylation status of one or more (e.g., 1, 2, 5, 10, 20, 50, 100, 200, 500, 1000, or more) of the plurality of biomarkers is indicative of a subject suffering severe MS (PDDS≥4) versus suffering from non-severe MS (PDDS<4). In some embodiments, the methylation status of one or more (e.g., 1, 2, 5, 10, 20, 50, 100, 200, 500, 1000, or more) of the plurality of biomarkers is indicative of a subject suffering non-severe MS (PDDS<4) versus suffering from severe MS (PDDS≥4). In some embodiments, the methylation status of one or more (e.g., 1, 2, 5, 10, 20, 50, 100, 200, 500, 1000, or more) of the plurality of biomarkers is indicative of a subject in active relapse of MS versus stable remission from MS. In some embodiments, the methylation status of one or more (e.g., 1, 2, 5, 10, 20, 50, 100, 200, 500, 1000, or more) of the plurality of biomarkers is indicative of a subject in stable remission from MS versus active relapse of MS. In some embodiments, the plurality of biomarkers are selected from the CpGs of Supplementary Table 3 (CpG Nos. 1-4360). In some embodiments, the plurality of biomarkers are selected from the DMRs of Supplementary Table 4 (DFR Nos. 1-1401). In some embodiments, the plurality of biomarkers are selected from the CpGs of Supplementary Table 7 (CpG Nos. 4361-5804). In some embodiments, the plurality of biomarkers are selected from the DMRs of Supplementary Table 8 (DFR Nos. 1402-1913).

In some embodiments, provided herein are methods of treating a subject comprising conducting a method of detecting the methylation status of a plurality of biomarkers in a sample comprising cell free DNA (cfDNA) by the methods described herein, and administering to the subject a treatment for MS.

In some embodiments provided herein are panels of biomarkers wherein each of the biomarkers is a methylation locus comprising at least a single CpG site and/or differentially methylated region (DMR) that is indicative of multiple sclerosis (MS) status. In some embodiments, the methylation status of one or more (e.g., 1, 2, 5, 10, 20, 50, 100, 200, 500, 1000, or more) of the plurality of biomarkers is indicative of a subject suffering from MS versus not suffering from MS. In some embodiments, the methylation status of one or more (e.g., 1, 2, 5, 10, 20, 50, 100, 200, 500, 1000, or more) of the plurality of biomarkers is indicative of a subject suffering from progressive MS (PMS) versus suffering from relapsing-remitting MS (RRMS). In some embodiments, the methylation status of one or more (e.g., 1, 2, 5, 10, 20, 50, 100, 200, 500, 1000, or more) of the plurality of biomarkers is indicative of a subject suffering from RRMS versus suffering from PMS. In some embodiments, the methylation status of one or more (e.g., 1, 2, 5, 10, 20, 50, 100, 200, 500, 1000, or more) of the plurality of biomarkers is indicative of a subject suffering from primary progressive MS (PPMS) versus suffering from secondary progressive MS (SPMS). In some embodiments, the methylation status of one or more (e.g., 1, 2, 5, 10, 20, 50, 100, 200, 500, 1000, or more) of the plurality of biomarkers is indicative of a subject suffering from SPMS versus suffering from PPMS. In some embodiments, the methylation status of one or more (e.g., 1, 2, 5, 10, 20, 50, 100, 200, 500, 1000, or more) of the plurality of biomarkers is indicative of a subject suffering severe MS (PDDS≥4) versus suffering from non-severe MS (PDDS<4). In some embodiments, the methylation status of one or more (e.g., 1, 2, 5, 10, 20, 50, 100, 200, 500, 1000, or more) of the plurality of biomarkers is indicative of a subject suffering non-severe MS (PDDS<4) versus suffering from severe MS (PDDS≥4). In some embodiments, the methylation status of one or more (e.g., 1, 2, 5, 10, 20, 50, 100, 200, 500, 1000, or more) of the plurality of biomarkers is indicative of a subject in active relapse of MS versus stable remission from MS. In some embodiments, the methylation status of one or more (e.g., 1, 2, 5, 10, 20, 50, 100, 200, 500, 1000, or more) of the plurality of biomarkers is indicative of a subject in stable remission from MS versus active relapse of MS. In some embodiments, the plurality of biomarkers are selected from the CpGs of Supplementary Table 3 CpG Nos. 1-4360). In some embodiments, the plurality of biomarkers are selected from the DMRs of Supplementary Table 4 (DFR Nos. 1-1401). In some embodiments, the plurality of biomarkers are selected from the CpGs of Supplementary Table 7 (CpG Nos. 4361-5804). In some embodiments, the plurality of biomarkers are selected from the DMRs of Supplementary Table 8 (DFR Nos. 1402-1913).

In some embodiments, the plurality of biomarkers in a panel or method herein comprises 5 or more biomarkers (e.g., >5, >10, >15, >20, >25, >30, >40, >50, >60, >70, >80, >90, >100, >150, >200, >250, >300, >400, >500, >750, >1000, >1500, >2000, or more). In some embodiments, the plurality of biomarkers in a panel or method herein comprises 5000 or fewer biomarkers (e.g., <5000, <4000, <3000, <2000, <1500, <1000, <750, <500, <400, <300, <250, <200, <150, <100, <90, <80, <70, <60, <50, or fewer).

### BRIEF DESCRIPTION OF THE DRAWINGS

FIG. 1. Experimental design.

FIG. 2A-F. Circulating cfDNA methylation and its inferred tissue-of-origin patterns distinguished MS from controls and among different MS subtypes. A. Scatter plots of −log 10 (FDR) vs mean methylation differences at differentially methylated CpGs between MS and controls (left panel), between progressive MS (PMS: PPMS or SPMS) and relapsing-remitting MS (RRMS) (middle panel), and between actively relapsing RRMS (A-RMSS) and stable remission RRMS (S-RMSS) (right panel). Colors indicate CpGs with FDR<0.1 and absolute mean methylation difference >0.1. B. Uniform Manifold Approximation and Projection (UMAP) representation of cfDNA methylation levels in DMRs could distinguish pwMS from controls and across MS subtypes. C. Enrichment and depletion of DMRs in different chromHMM states (that were previously computed from GM12878 cells in the Epigenome Roadmap project) when comparing MS vs controls or when comparing between MS subtypes. D. Gene ontology enrichment analysis of DMRs between MS and non-MS controls with prioritization by g: profile. (See FIG. 6 for the gene ontology enrichment analysis of DMRs between PMS and RRMS as well as between A-RMSS and S-RRMS.) E. Differences in cell death contribution from brain Neuron (left panel), T cells (middle panel), and Monocytes (right panel) when comparing MS with controls and when comparing across MS subtypes. F. Receiver operating characteristic (ROC) curve by DMR only (red), Tissue-of-origin only (TOO, orange), NfL only (grey), and GFAP only (black) to differentiate MS and controls (left panel), PMS and RRMS (middle panel), and A-RRMS and S-RRMS (right panel). NfL and GFAP were measured from a subset of the participants from the same cohort as approximate historical benchmarks.

FIG. 3A-F. Circulating cfDNA methylation and its inferred tissue-of-origin patterns distinguished individuals with different disability levels. A. Scatter plots of −log 10 (FDR) vs mean methylation differences at differentially methylated CpGs between two disability severity levels based on the requirement for ambulatory assistance around sample collection: higher PDDS scores (severe disability, PDDS≥4) vs lower PDDS scores (non-severe, i.e., normal-mild-moderate disability, PDDS<4). Colors indicated CpGs with FDR<0.1 and absolute mean methylation difference >0.1. PDDS score (i.e., patient determined disease steps) indicated patient-reported ambulatory disability status. B. Uniform Manifold Approximation and Projection (UMAP) representation of cfDNA methylation level in DMRs could distinguish pwMS based on the binary threshold of patient-reported disability (PDDS≥4 vs <4). C. Enrichment and depletion of DMRs in different chromHMM states (that were previously computed from GM12878 cells in the Epigenome Roadmap project) when comparing between pwMS with severe disability (PDDS≥4) vs non-severe disability (PDDS<4). D. Gene ontology enrichment analysis of DMRs between pwMS with severe disability (PDDS≥4) vs non-severe disability (PDDS<4) with prioritization by g: profile. E. Differences in cell death contribution from Monocytes when comparing between pwMS with severe disability (PDDS≥4) vs non-severe disability (PDDS<4). (See FIG. 12 for various Epithelial cell types.) F. Receiver operating characteristic (ROC) curve by DMR only (red), Tissue-of-origin only (TOO, orange), NfL only (grey), and GFAP only (black) to distinguish MS patients based on patient-reported disability: Most Severe Disability (PDDS≥6) vs Least Severe (i.e., Normal-Mild, PDDS≤1). NfL and GFAP were measured from a subset of the participants from the same cohort as approximate historical benchmarks.

FIG. 4A-C. Baseline circulating DNA methylation profiles informed future disability severity trajectory. A. Representative plots for the patient-reported disability severity (i.e., PDDS) trajectories over time stratified according to baseline circulating cfDNA methylation level (high, ≥median, red; low, <median, green) at one representative prognostic genomic region. The group with higher methylation levels showed increasing trajectory in patient-reported disability severity over time. The p-value was corrected for multiple testing (Bonferroni). The shaded area represented the 95% confidence interval. PDDS, patient determined disease steps. B. Receiver operating characteristic (ROC) curve using the baseline cfDNA methylation level at 5,392 prognostic regions to predict the binary status of patient-reported disability severity at 1,000 days after the blood sample collection: the most severe disability (PDDS≥6) vs the least (i.e., normal-mild) disability (PDDS≤1). C. Progression-free survival curve using the risk score calculated from the weighted sum of baseline circulating cfDNA methylation level at the top 100 most informative prognostic regions. The prognostic regions were categorized based on the product of absolute value of effect size (time×biomarker interaction term) and −log 10 FDR (time×biomarker interaction term) in each region. The p-value was calculated by the log-rank test. MBPRS score (high, ≥median, red; low, <median, blue) stratified patients into significantly different disability progression trajectories (i.e., higher MBPRS predicted worse progression-free survival). MBPRS, Methylation-based progression risk score.

FIG. 5A-B. Variance explained by the top singular value decompositions (SVDs). A. Absolute (left) and proportion (right) of variance explained by the top SVDs for the cfDNA methylation level from differentially methylated regions (DMRs) that were called across MS subtypes. B. Absolute (left) and proportion (right) of variance explained by the top SVDs for the cfDNA methylation level from DMRs that were called between the two disability severity levels based on the requirement of ambulatory assistance around sample collection: higher PDDS scores (severe disability, PDDS≥4) and lower PDDS scores (non-severe, i.e., normal-mild-moderate disability, PDDS<4). PDDS, patient determined disease steps.

FIG. 6A-B. Gene ontology analysis on differentially methylated regions (DMRs). A. PMS vs RRMS. B. A-RRMS vs S-RRMS. PMS, progressive MS; RRMS, relapsing-remitting MS; A-RRMS, actively relapsing RRMS; S-RRMS, stable remission RRMS.

FIG. 7A-B. Motif enrichment at differentially methylated regions (DMRs). A. MS vs controls. B. PMS vs RRMS. C. A-RRMS vs S-RRMS. Enrichment was calculated by g: profile. PMS, progressive MS; RRMS, relapsing-remitting MS; A-RRMS, actively relapsing RRMS; S-RRMS, stable remission RRMS.

FIG. 8. Tissue-of-origin estimated from different MS subtypes and non-MS controls. One-side Mann-Whitney U test was performed between every two groups of participants (e.g., PMS vs A-RRMS, or PMS vs A-RRMS+S-RRMS+Control). The side was determined if the median was larger or smaller between the groups. PMS, progressive MS; RRMS, relapsing-remitting MS; A-RRMS, actively relapsing RRMS; S-RRMS, stable remission RRMS. *, p<0.05; **, p<0.01.

FIG. 9A-B. Simulation results on the variation of identifying tissue-of-origin at different coverages of cfDNA WGBS. A. Tissue fractions at different downsampled coverages across tissues and cell types. B. The standard deviation of tissue fraction at different downsampled coverages across tissues and cell types.

FIG. 10. Gene ontology analysis on differentially methylated regions (DMRs) between disability severity groups. Two disability severity groups were categorized based on the requirement for ambulatory assistance around sample collection: higher PDDS scores (severe disability, PDDS≥4) and lower PDDS scores (non-severe, i.e., normal-mild-moderate disability, PDDS<4). PDDS, patient determined disease steps.

FIG. 11. Motif enrichment on differentially methylated regions (DMRs) between disability severity groups. Two disability severity groups were categorized based on the requirement for ambulatory assistance around sample collection: higher PDDS scores (severe disability, PDDS≥4) and lower PDDS scores (non-severe, i.e., normal-mild-moderate disability, PDDS<4). PDDS, patient determined disease steps.

FIG. 12. Tissue-of-origin estimated between disability severity groups. Two disability severity groups were categorized based on the requirement for ambulatory assistance around sample collection: higher PDDS scores (severe disability, PDDS≥4) and lower PDDS scores (non-severe, i.e., normal-mild-moderate disability, PDDS<4). One-side Mann-Whitney U test was performed between groups of patients. The side was determined if the median was larger or smaller between the groups. PDDS, patient determined disease steps. *, p<0.05.

FIG. 13. Additional representative plots for the patient-reported disability severity trajectories over time stratified according to baseline cfDNA methylation level. Baseline cfDNA methylation level (high, ≥median, red; vs low, <median, green) at two additional representative prognostic genomic regions. The group with lower methylation levels (left panel) and the group with higher methylation levels (right panel) showed increasing trajectory in patient-reported disability severity over time, respectively. The p-value was corrected for multiple testing (Bonferroni). The shaded area represented the 95% confidence interval. PDDS, patient determined disease steps.

FIG. 14A-B. Log-rank test p value from two different sets of control regions. A. Progression-free survival curve using the risk score calculated from the weighted sum of baseline cfDNA methylation level at 100 least informative prognostic regions. The prognostic regions were categorized based on the product of absolute value of effect size (time×biomarker interaction term) and −log 10 FDR (time×biomarker interaction term) in each region. The p value was calculated by the log-rank test. This “control” MBPRS score (high, ≥median, red; low, <median, blue) did not reach statistical significance via log rank test despite visible separation of survival curves. MBPRS, Methylation based progression risk score. B. The distribution of permutated p-values calculated from the matched random control regions (matched chromosomes and region sizes). MBPRS for each patient were calculated using the original effect size (from the top 100 regions) and baseline cfDNA methylation level from matched random regions. For each iteration, the log-rank test p-value was calculated between the two groups of patients with a different MBPRS score. The process was repeated 1,000 times to obtain the p-value distribution. The observed log-rank test p-value from the top 100 informative prognostic regions (FIG. 4C) was marked as the red line here.

FIG. 15. Quality measurement of 200 cfDNA WGBS data. Upper (left): Library complexity. Upper (middle): Boxplot of the number of reads in different quality category. Upper (right): Boxplot of bisulfite incomplete conversion rates. Lower: Barplot of the number of reads in different quality category across all the samples.

FIG. 16. The number and volcano plot of DMCs and DMRs between MS and non-MS controls and between MS subtypes.

FIG. 17. DMRs distinguish MS subtypes and non-MS controls. Left: heatmap of methylation level in the DMRs. Middle: umap of DNA methylation level in merged DMRs, colored by MS subtypes. Right: umap of DNA methylation level in merged DMRs, colored by disability severity (PDDS score).

FIG. 18. DMRs are enriched in gene regulatory regions. Left: chromHMM states heatmap enrichment of DMRs discovered in pilot study. Right: heatmap enrichment of DMRs discovered in the current study.

FIG. 19. DMRs are enriched in biological processes. Left: gene ontology heatmap enrichment of DMRs discovered in pilot study. Right: heatmap enrichment of DMRs discovered in the current study.

FIG. 20. TOO with more reference methylome from single-cell WGBS in human brain. Left: new DNA methylation reference map. Right: TOO results in the current study using the new reference methylome map.

FIG. 21. TOO differences across MS subtypes. Left: Neuron cell death difference across MS subtypes. Right (upper): monocyte cell death difference across MS subtypes. Right (down): granulocyte cell death difference across MS subtypes.

FIG. 22. Machine learning classifier. Left: ROC curve (PMS vs. RRMS) in the pilot study. Middle: ROC curve of using DNA methylation in the DMRs found in the training folds in the current study (PMS vs. RRMS). Right: ROC curve of using DNA methylation in the DMRs found in the training folds and clinical variables in the current study (PMS vs. RRMS).

FIG. 23. Machine learning classifier to distinguish PPMS from SPMS using DNA methylation in the DMRs found in the training folds and clinical variables in the current study.

FIG. 24. The number and volcano plot of DMCs and DMRs between severe and non-severe MS patients.

FIG. 25. DMRs distinguish MS patients with different severities. Left: umap of DNA methylation level in merged DMRs, colored by PDDS score. Right: heatmap of methylation level in the DMRs.

FIG. 26. DMRs are enriched in gene regulatory regions. Left: chromHMM states heatmap enrichment of DMRs discovered in pilot study. Right: heatmap enrichment of DMRs discovered in the current study.

FIG. 27. DMRs between severe and non-severe MS patients are enriched in biological processes.

FIG. 28. Machine learning classifier to distinguish most severe and least severe MS patients. Left: ROC curve in the pilot study. Right: ROC curve of using DNA methylation in the DMRs found in the training folds in the current study.

FIG. 29. TOO differences between MS patients with different disease severity. Upper (left): Monocyte cell death. Upper (Middle): Neuron cell death. Lower (left): Monocyte cell death across more different PDDS categories. Lower (middle): Oligodendrocyte cell death across more different PDDS categories. Lower (right): Microglia cell death across more different PDDS categories.

FIG. 30. Relapse-free survival curve stratified by MS patients with different methylation based prognostic score (MBPRS). P value is calculated by log-rank test. Patients are stratified by the median of MBPRS value.

FIG. 31. Relapse-free survival curve stratified by the median of cell death values. P value is calculated by log-rank test.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a biomarker” is a reference to one or more biomarkers and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “Multiple Sclerosis” or “MS” refers to a chronic disorder of the central nervous system characterized by immune-mediated demyelination, inflammation, neuroaxonal injury, or a combination thereof, resulting in focal or diffuse neurological dysfunction, and includes all clinically recognized forms and variants thereof.

As used herein, the term “subject” or “patient” refers to a human individual who is suspected of having, is at risk of developing, has been diagnosed with, or is undergoing monitoring or treatment for MS, including individuals presenting with neurological symptoms, radiographic findings, or biomarkers suggestive of demyelinating disease.

As used herein, the term “clinically isolated syndrome” or “CIS” refers to a first clinical episode of neurological symptoms attributable to demyelination of the central nervous system, lasting at least 24 hours, and not yet meeting formal diagnostic criteria for MS.

As used herein, the term “radiologically isolated syndrome” or “RIS” refers to the incidental detection of radiographic features consistent with demyelinating lesions of MS in the absence of overt clinical symptoms.

As used herein, the term “relapsing-remitting multiple sclerosis” or “RRMS” refers to a disease course characterized by discrete clinical relapses followed by periods of partial or complete remission, without continuous progression of neurological disability between relapses.

As used herein, the term “secondary progressive multiple sclerosis” or “SPMS” refers to a disease course that follows an initial relapsing-remitting phase and is characterized by gradual, sustained worsening of neurological function with or without superimposed relapses.

As used herein, the term “primary progressive multiple sclerosis” or “PPMS” refers to a disease course characterized by progressive neurological deterioration from disease onset, in the absence of clearly defined relapses or remissions.

As used herein, the term “disease activity” refers to evidence of ongoing inflammatory or demyelinating processes, as determined by clinical relapse, radiographic findings, biomarker measurements, or combinations thereof.

As used herein, the term “disease progression” refers to sustained worsening of neurological function or disability independent of acute inflammatory relapses.

As used herein, the term “dissemination in space” refers to the presence of pathological findings consistent with demyelination in two or more anatomically distinct regions of the central nervous system.

As used herein, the term “dissemination in time” refers to evidence that demyelinating events have occurred at different points in time, as determined by clinical history, imaging, laboratory findings, or combinations thereof.

As used herein, the term “diagnostic criterion” refers to a predefined rule, threshold, or framework used to establish or support a diagnosis of MS, including but not limited to criteria based on clinical findings, imaging, cerebrospinal fluid analysis, or biomarkers.

As used herein, the term “biomarker” refers to any measurable biological characteristic that correlates with the presence, activity, severity, progression, prognosis, or treatment response of MS, including molecular, cellular, genetic, proteomic, or imaging-derived markers.

As used herein, the term “severity” refers to the extent of neurological impairment, disability, disease burden, or structural damage attributable to MS, as assessed by clinical examination, disability scales, imaging metrics, biomarkers, or combinations thereof.

As used herein, the terms “treat,” “treating,” and “treatment” include inhibiting the pathological condition, disorder, or disease (e.g., MS), e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.), intracranially, and the like.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “diagnosis” refers to the detection, determination, or recognition of a health status or condition of an individual on the basis of one or more signs, symptoms, data, or other information pertaining to that individual. The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present disclosure.

As used herein, the term “cell-Free DNA” (“cfDNA”) refers to DNA that has been released from cells as a result of natural cell death/turnover etc. or as a result of disease processes. The cfDNA is released into the circulation and rapidly broken down into DNA fragments and can ultimately end up in other body fluids. The techniques for the harvesting of cfDNA from the blood and other body fluids are well-known in the arts (Li Y et al. Size separation of circulatory DNA in maternal plasma permits ready detection of fetal DNA polymorphisms. Clin Chem 2004; 50:1002-1011; Zimmerman B et al. Noninvasive prenatal aneuploidy testing of chromosomes 13, 18, 21, X, and Y, using targeted sequencing of polymorphic loci. Prenat Diagn 2012; 32:1233-41).

### DETAILED DESCRIPTION

Provided herein are circulating cell-free DNA methylation profiles and methods of use thereof as noninvasive multiple sclerosis biomarkers.

Circulating cell-free DNA (cfDNA) from peripheral blood has emerged as a non-invasive, accessible, and cost-effective biomarker for multiple diseases. 17-20 It is well suited for capturing complex pathogenesis underlying chronic autoimmune diseases such as MS, which involves peripheral immune activation, inflammatory demyelination and CNS neurodegeneration. In people with MS (pwMS), cfDNA can originate from multiple cell types undergoing apoptosis or necrosis, including T cells, B cells, and monocyte cells of the peripheral immune system as well as oligodendrocytes and neurons of the CNS. Distinct cfDNA methylation patterns at individual genomic regions differentiated pwMS from controls.23-25 Genome-wide and tissue-specific studies reported widespread aberrant DNAmethylation patterns in the peripheral immune system and CNS of pwMS,26-32 which collectively add to the circulating cfDNA pool and justify a strategy of concurrently profiling the peripheral immune system and CNS. Given the cell-type specificity of DNA methylation, cfDNA methylation signatures could indicate the tissues of origin, enabling a detailed analysis of underlying pathological processes.

It was contemplated that circulating cfDNA methylation profiles would have clinical utility in subtyping MS, monitoring disability severity, and predicting disease progression and could eventually complement current methods (e.g., MRI, existing blood biomarkers). For instance, a greater proportion of CNS-derived cfDNA might suggest progressive MS subtypes or worse neurodegeneration, whereas elevated peripheral immune cells-derived cfDNA may indicate acute inflammatory disease activity. Clinical implementation of cfDNA methylation profiles could facilitate earlier initiation or discontinuation of subtype-specific DMTs, more frequent monitoring, and iterative prognosis adjustments tailored to the evolving patient profiles.

To demonstrate the clinical utility, it is crucial to examine the extent to which biomarkers perform in relevant clinical contexts using affordable methods. Low-coverage (~1.5×) whole-genome bisulfite sequencing (WGBS) was performed on 75 plasma samples from pwMS and controls in a well-characterized real-world prospective clinic cohort with longitudinal disability outcome measurements. The genome-wide differences in cfDNA methylation signature were assessed and its inferred tissue-of-origin between pwMS and controls, across MS subtypes, and between pwMS having severe vs non-severe disability status. Using cfDNA methylation profiles and machine learning, pwMS was differentiated from controls, across MS subtypes, and between disability severity levels. A linear mixed model was deployed to identify “prognostic regions” in baseline cfDNA methylation profile associated with subsequent disability progression after confounder adjustment and generated a cfDNA methylation-based progression risk score (MBPRS) using these prognostic regions to stratify patients with differential progression risks.

In this study to assess the potential clinical utility of cfDNA methylation profiles as MS biomarkers for multiple clinical contexts, low-coverage (~1.5×) WGBS data was generated from 75 plasma cfDNA samples, including MS subtypes and non-MS controls. Using a beta-binomial regression model, thousands of differentially methylated CpGs and hundreds of DMRs were identified despite challenges due to low-coverage sequencing. cfDNA methylation levels at these DMRs and their inferred tissue-of-origin patterns distinguished pwMS from controls, differentiated across MS subtypes, and correlated with disability severity. Putative gene-regulatory functions were further identified for these DMRs and distinct tissue-of-origin patterns among participant groups. Baseline cfDNA methylation profiles showed promise in predicting future disability status within a 3-year evaluation window. Finally, a baseline summary score (i.e., MBPRS) stratified patients into groups with differential disability progression trajectories. This study has several strengths and novelties. First, this was the first whole-genome investigation of plasma cfDNA methylation and its tissue-of-origin pattern as biomarkers in MS clinical contexts to our knowledge. The tissue-of-origin patterns among MS subtypes and pwMS with differential disability severity not only confirmed previous knowledge but also highlighted the less-appreciated roles of other peripheral immune cell types in MS pathogenesis for future validation. Second, this study demonstrated the potential of circulating cfDNA methylation profile and its tissue-of-origin pattern as biomarkers to concurrently diagnose MS, classify MS subtype, evaluate disability severity status, and predict long-term disability progression trajectory. Our preliminary evidence suggested that circulating cfDNA methylation profile as a single assay for MS diagnosis, subtyping, and monitoring and prognosis could potentially complement established blood biomarkers such as NfL and GFAP. Third, this study leveraged samples collected from a well-characterized real-world prospective clinic cohort with a disability outcome measure (i.e., PDDS) already deployed longitudinally in routine clinical care. The novelty lies in the use of longitudinal clinical outcome data to identify prognostic regions in baseline cfDNA methylation profile and to potentially inform individualized clinical guidance and eventually improve patient outcomes.

Experiments conducted during development of embodiments herein demonstrated circulating cfDNA methylation profiles as MS biomarkers for multiple clinical contexts.

In some embodiments, provided herein are biomarkers (e.g., CpG sites and/or DMRs) and methods of analysis thereof for diagnosing MS, determining susceptibility to MS, subtyping MS, determining the severity of MS, etc., including steps of obtaining a sample (e.g., blood sample) from a subject and extracting cell-free (cf) DNA from the sample as extracted cfDNA. The degree of methylation in one or a plurality of CpGs and/or DMRs in the extracted cfDNA is identified. The target subject is diagnosed and/or prognosed for MS, MS subtype, MS severity, etc. if the amount of methylation of one or more of the biomarkers differs from the amount of methylation established in control subjects to a statistically significant degree.

In some embodiments, methylation biomarkers herein find using in diagnosing a subject with MS. In some embodiments, assessment of biomarkers herein finds use in distinguishing between MS and non-MS for a subject at risk or suspected of suffering from MS. In some embodiments, a subject identified as suffering from MS using the biomarkers described herein (alone or in conjunction with one or more additional diagnostic criteria) is treated for MS.

In some embodiments, methylation biomarkers herein find use in subtyping MS in a subject. In some embodiments, biomarkers are provided herein for distinguishing between progressive MS and relapsing-remitting MS. In some embodiments, methods are provided for diagnosing a subject with progressive MS (e.g., PPMS, SPMS, etc.). In some embodiments, methods are provided for diagnosing a subject with RRMS. In some embodiments, a subject identified as suffering from RRMS using the biomarkers described herein (alone or in conjunction with one or more additional diagnostic criteria) is treated for RRMS. In some embodiments, a subject identified as suffering from SPMS using the biomarkers described herein (alone or in conjunction with one or more additional diagnostic criteria) is treated for SPMS. In some embodiments, a subject identified as suffering from PPMS using the biomarkers described herein (alone or in conjunction with one or more additional diagnostic criteria) is treated for PPMS.

In some embodiments, methylation biomarkers herein find use in evaluating the severity of MS in a subject. In some embodiments, biomarkers are provided for distinguishing between severe (PDDS≥4) vs non-severe (PDDS<4) MS. In some embodiments, methods are provided for assessing the severity of MS in a subject using the biomarkers herein. In some embodiments, a subject identified as suffering from severe MS using the biomarkers described herein (alone or in conjunction with one or more additional diagnostic criteria) is treated for severe MS. In some embodiments, a subject identified as suffering from non-severe MS using the biomarkers described herein (alone or in conjunction with one or more additional diagnostic criteria) is treated for non-severe MS.

In some embodiments, methylation biomarkers herein find use in evaluating whether a subject is suffering from active relapse or stable remission. In some embodiments, biomarkers are provided for distinguishing between active relapse or stable remission. In some embodiments, methods are provided for assessing whether a subject is in active relapse or stable remission. In some embodiments, a subject identified as in active relapse from MS using the biomarkers described herein (alone or in conjunction with one or more additional diagnostic criteria) is treated for active relapse from MS. In some embodiments, a subject identified as in stable remission from MS using the biomarkers described herein (alone or in conjunction with one or more additional diagnostic criteria) is treated for stable remission from MS.

In various embodiments, a methylation biomarker of the present disclosure used for assessment of MS status, MS subtype, MS prognosis, MS severity, and/or of the treatment of MS. Such methylation biomarkers may include CpGs and/or DMRs, such as those provided herein as in Supplementary Tables 3, 4, 7, and/or 8. These CpG sites and DMRs are useful in the assessment and subtyping, etc. of MS in a subject.

Supplementary Table 3 lists differential CpG sites in cfDNA WGBS comparing MS and controls and comparing across MS subtypes.

In some embodiments, the methylation status of one or more (e.g., 1, 2, 5, 10, 20, 50, 100, 200, 500, 1000, 2000, 2107, or ranges therebetween) of CpG Nos. 1-2107 (Supplementary Table 3) differentiate MS from non-MS. In some embodiments, methods comprise determining the methylation status of any combination of one or more (e.g., 1, 2, 5, 10, 20, 50, 100, 200, 500, 1000, 2000, 2107, or ranges therebetween) of CpG Nos. 1-2107 (Supplementary Table 3), comparing the results to control(s) (e.g., for the subject or a population) or threshold methylation values, and making a MS-status determination for the subject based thereon (e.g., pwMS vs. no MS). In some embodiments, a biomarker panel is provided comprising any combination of one or more (e.g., 1, 2, 5, 10, 20, 50, 100, 200, 500, 1000, 2000, 2107, or ranges therebetween) of CpG Nos. 1-2107 (Supplementary Table 3).

In some embodiments, the methylation status of one or more (e.g., 1, 2, 5, 10, 20, 50, 100, 200, 500, 1000, 1191, or ranges therebetween) of CpG Nos. 2108-3298 (Supplementary Table 3) differentiate PMS from RRMS. In some embodiments, methods comprise determining the methylation status of any combination of one or more (e.g., 1, 2, 5, 10, 20, 50, 100, 200, 500, 1000, 1191, or ranges therebetween) of CpG Nos. 2108-3298 (Supplementary Table 3), comparing the results to control(s) (e.g., for the subject or a population) or threshold methylation values, and making a MS-status determination for the subject based thereon (e.g., PMS vs. RRMS). In some embodiments, a biomarker panel is provided comprising any combination of one or more (e.g., 1, 2, 5, 10, 20, 50, 100, 200, 500, 1000, 1191, or ranges therebetween) of CpG Nos. 2108-3298 (Supplementary Table 3).

In some embodiments, the methylation status of one or more (e.g., 1, 2, 5, 10, 20, 50, 100, 200, 500, 1000, 1061, or ranges therebetween) of CpG Nos. 3299-4360 (Supplementary Table 3) differentiate PPMS from SPMS. In some embodiments, methods comprise determining the methylation status of any combination of one or more (e.g., 1, 2, 5, 10, 20, 50, 100, 200, 500, 1000, 1061, or ranges therebetween) of CpG Nos. 3299-4360 (Supplementary Table 3), comparing the results to control(s) (e.g., for the subject or a population) or threshold methylation values, and making a MS-status determination for the subject based thereon (e.g., PMS vs. RRMS). In some embodiments, a biomarker panel is provided comprising any combination of one or more (e.g., 1, 2, 5, 10, 20, 50, 100, 200, 500, 1000, 1061, or ranges therebetween) of CpG Nos. 3299-4360 (Supplementary Table 3).

Supplementary Table 4 lists DMRs in cfDNA WGBS comparing MS and controls and comparing across MS subtypes.

In some embodiments, the methylation status of one or more (e.g., 1, 2, 5, 10, 20, 50, 100, 200, 500, 1000, 2000, 2107, or ranges therebetween) of DMR Nos. 1-2107 (Supplementary Table 4) differentiate MS from non-MS. In some embodiments, methods comprise determining the methylation status of any combination of one or more (e.g., 1, 2, 5, 10, 20, 50, 100, 200, 500, 1000, 2000, 2107, or ranges therebetween) of DMR Nos. 1-2107 (Supplementary Table 4), comparing the results to control(s) (e.g., for the subject or a population) or threshold methylation values, and making a MS-status determination for the subject based thereon (e.g., pwMS vs. no MS). In some embodiments, a biomarker panel is provided comprising any combination of one or more (e.g., 1, 2, 5, 10, 20, 50, 100, 200, 500, 1000, 2000, 2107, or ranges therebetween) of DMR Nos. 1-2107 (Supplementary Table 4).

In some embodiments, the methylation status of one or more (e.g., 1, 2, 5, 10, 20, 50, 100, 200, 500, 1000, 1191, or ranges therebetween) of DMR Nos. 2108-3298 (Supplementary Table 4) differentiate PMS from RRMS. In some embodiments, methods comprise determining the methylation status of any combination of one or more (e.g., 1, 2, 5, 10, 20, 50, 100, 200, 500, 1000, 1191, or ranges therebetween) of DMR Nos. 2108-3298 (Supplementary Table 4), comparing the results to control(s) (e.g., for the subject or a population) or threshold methylation values, and making a MS-status determination for the subject based thereon (e.g., PMS vs. RRMS). In some embodiments, a biomarker panel is provided comprising any combination of one or more (e.g., 1, 2, 5, 10, 20, 50, 100, 200, 500, 1000, 1191, or ranges therebetween) of DMR Nos. 2108-3298 (Supplementary Table 4).

In some embodiments, the methylation status of one or more (e.g., 1, 2, 5, 10, 20, 50, 100, 200, 387, or ranges therebetween) of DMR Nos. 3299-4360 (Supplementary Table 4) differentiate PPMS from SPMS. In some embodiments, methods comprise determining the methylation status of any combination of one or more (e.g., 1, 2, 5, 10, 20, 50, 100, 200, 387, or ranges therebetween) of DMR Nos. 3299-4360 (Supplementary Table 4), comparing the results to control(s) (e.g., for the subject or a population) or threshold methylation values, and making a MS-status determination for the subject based thereon (e.g., PPMS vs. SPMS). In some embodiments, a biomarker panel is provided comprising any combination of one or more (e.g., 1, 2, 5, 10, 20, 50, 100, 200, 387, or ranges therebetween) of DMR Nos. 3299-4360 (Supplementary Table 4).

Supplementary Table 7 lists differential CpG sites in cfDNA WGBS comparing MS patients with higher vs lower patient-reported disability (PDDS≥4 vs <4).

In some embodiments, the methylation status of one or more (e.g., 1, 2, 5, 10, 20, 50, 100, 200, 500, 1000, 1444, or ranges therebetween) of CpG Nos. 4361-5804 (Supplementary Table 7) differentiate severe MS disability from non-severe MS disability. In some embodiments, methods comprise determining the methylation status of any combination of one or more (e.g., 1, 2, 5, 10, 20, 50, 100, 200, 500, 1000, 1444, or ranges therebetween) of CpG Nos. 4361-5804 (Supplementary Table 7), comparing the results to control(s) (e.g., for the subject or a population) or threshold methylation values, and making a MS-status determination for the subject based thereon (e.g., severe MS disability vs. non-severe MS disability). In some embodiments, a biomarker panel is provided comprising any combination of one or more (e.g., 1, 2, 5, 10, 20, 50, 100, 200, 500, 1000, 1444, or ranges therebetween) of CpG Nos. 4361-5804 (Supplementary Table 7).

Supplementary Table 8 lists DMRs in cfDNA WGBS comparing MS patients with higher vs lower patient-reported disability (PDDS≥4 vs <4).

In some embodiments, the methylation status of one or more (e.g., 1, 2, 5, 10, 20, 50, 100, 200, 500, 512, or ranges therebetween) of DMR Nos. 1402-1913 (Supplementary Table 8) differentiate severe MS disability from non-severe MS disability. In some embodiments, methods comprise determining the methylation status of any combination of one or more (e.g., 1, 2, 5, 10, 20, 50, 100, 200, 500, 512, or ranges therebetween) of DMR Nos. 1402-1913 (Supplementary Table 8), comparing the results to control(s) (e.g., for the subject or a population) or threshold methylation values, and making a MS-status determination for the subject based thereon (e.g., severe MS disability vs. non-severe MS disability). In some embodiments, a biomarker panel is provided comprising any combination of one or more (e.g., 1, 2, 5, 10, 20, 50, 100, 200, 500, 512, or ranges therebetween) of DMR Nos. 1402-1913 (Supplementary Table 8).

Beyond the limits described herein, the combinations of CpGs and DMRs that find use in the methods and biomarker panels are not otherwise limited. Any suitable number of biomarkers in any suitable combination may find use in embodiments herein.

In some particular embodiments, combinations of the CpGs and/or DMRs herein are useful in the detection of MS, determination of MS subtype, determination of MS severity, determining a treatment, in the treatment of MS, determining a long-term prognosis, etc. CpGs, DMRs, portions of DMRs, and combinations thereof specifically identified in Supplementary Tables 3, 4, 7, and 8 are useful for assessing MS in a subject and/or providing treatment of MS. In certain embodiments, a methylation biomarker of the present disclosure used for assessing MS is selected from a CpG and/or methylation locus that is or includes at least a portion of a DMR listed in Supplementary Tables 3, 4, 7, and 8. In certain embodiments, a methylation locus that is or includes at least a portion of a DMR listed in Supplementary Tables 4 or 8 is particularly useful as a MS marker.

In some embodiments, said methylation biomarker can be or include a single methylation locus. In some embodiments, a methylation biomarker can be or include two or more methylation loci. In some embodiments, a methylation biomarker can be or include a single differentially methylated region (DMR) (e.g., (i) a DMR selected from those listed in Supplementary Table 4 or 8, (ii) a DMR that encompasses a DMR selected from those listed in Supplementary Table 4 or 8, (iii) a DMR that overlaps with one or more DMRs selected from those listed in Supplementary Table 4 or 8, or (iv) a DMR that is a portion of a DMR selected from those listed in Supplementary Table 4 or 8). In some embodiments, a methylation locus can be or include two or more DMRs (e.g., two, three, four, five, six, seven, eight, nine, ten, eleven, twelve, thirteen, fourteen, fifteen, twenty, thirty, forty, fifty, sixty, seventy, eighty, ninety, one hundred, two hundred, three hundred, four hundred, five hundred, one thousand, or more DMRs selected from those listed in Supplementary Table 4 or 8, or two, three, four, five, six, seven, eight, nine, ten, eleven, twelve, thirteen, fourteen, fifteen, twenty, thirty, forty, fifty, sixty, seventy, eighty, ninety, one hundred, two hundred, three hundred, four hundred, five hundred, one thousand, or more DMRs, each of which overlap with and/or encompass a DMR selected from those listed in Supplementary Table 4 or 8). In some embodiments, a methylation biomarker can be or include a single methylation site (e.g., a single CpG site, a methylated cytosine residue). In some embodiments, a methylation biomarker comprises one or more differential CpG sites of Supplementary Table 3. In some embodiments, a methylation biomarker comprises one or more DMRs of Supplementary Table 4. In some embodiments, a methylation biomarker comprises one or more differential CpG sites of Supplementary Table 7. In some embodiments, methylation biomarker comprises one or more DMRs of Supplementary Table 8. In other embodiments, a methylation biomarker can be or include two or more methylation sites. In some embodiments, a methylation locus can include two or more DMRs and further include DNA regions adjacent to one or more of the included DMRs.

In some instances, a methylation locus is or includes a gene. In some instances, a methylation locus may be or include a differential CpG site in Supplementary Table 3 or 7 which are not currently associated with any known gene. In some instances, a methylation locus may be or include portions of a DMR provided in Supplementary Table 4 or 8 which are not currently associated with any known gene.

Those of skill in the art will appreciate that a methylation locus identified as a methylation biomarker need not necessarily be assayed in a single experiment, reaction, or amplicon. A single methylation locus identified as a biomarker herein can be assayed, e.g., in a method including separate amplification (or providing oligonucleotide primers and conditions sufficient for amplification of) of one or more distinct or overlapping DNA regions within a methylation locus, e.g., one or more distinct or overlapping DMRs. Those of skill in the art will further appreciate that a methylation locus identified as a methylation biomarker need not be analyzed for methylation status of each nucleotide, nor each CpG, present within the methylation locus. Rather, a methylation locus that is a methylation biomarker may be analyzed, e.g., by analysis of a single DNA region within the methylation locus, e.g., by analysis of a single DMR within the methylation locus.

DMRs of the present disclosure can be a methylation locus or include a portion of a methylation locus. In some instances, a DMR is a DNA region with a methylation locus that is, e.g., 1 to 5,000 bp in length. In various embodiments, a DMR is a DNA region with a methylation locus that is equal to or less than 5,000 bp, 4,000 bp, 3,000 bp, 2,200 bp, 2,101 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp in length, covering at least 1 methylated CpG. In some embodiments, a DMR covers at least 1, at least 2, at least 3, at least 4, at least 5, at least 6, at least 7, at least 8, or at least 9 CpGs. In some embodiments, a DMR is greater than 20 bp, 50 bp, 100 bp, 121 bp, 200 bp, or greater in length.

Methylation biomarkers, including without limitation methylation loci and DMRs provided herein, can include at least one methylation site that is a biomarker of MS status (e.g., pwMS vs healthy) MS subtype (e.g., CIS, RRMS, SPMS, PPMS, etc.), disability severity status (e.g., PDDS), long-term disability progression trajectory, etc. In some embodiments, provided herein are multiple (e.g., 2, 3, 4, 5, 10, 20, 50, 100, 200, 500, 1000, or more) methylation biomarkers (e.g., CpGs, DMRs, etc.) that collectively provide one or more of MS status (e.g., pwMS vs healthy) MS subtype (e.g., CIS, RRMS, SPMS, PPMS, etc.), disability severity status (e.g., PDDS), long-term disability progression trajectory, etc.

For clarity, those of skill in the art will appreciate that the term “methylation biomarker” is used broadly, such that a methylation biomarker can include one or more methylation loci within a single DMR, and each methylation locus is also itself a methylation biomarker. Moreover, a single DMR can contain multiple methylation biomarkers. A methylation biomarker can be a subset of a single DMR, but a single methylation loci cannot span across multiple DMRs. As provided herein, a methylation locus can be any of one or more methylation loci each of which methylation loci is, includes, or is a portion of a gene (or specific DMR) identified in the Supplementary Tables herein (e.g., Supplementary Tables 3, 4, 7, or 8).

In various embodiments, a methylation biomarker can be or include one or more individual nucleotides (e.g., a single individual cytosine residue in the context of a CpG) or a plurality of individual cytosine residues (e.g., of a plurality of CpGs) present within one or more methylation loci (e.g., one or more DMRs) provided herein. Thus, in certain embodiments a methylation biomarker is or includes methylation status of a plurality of individual methylation sites.

In various embodiments, a methylation biomarker is, includes, or is characterized by change in methylation status that is a change in the methylation of one or more methylation sites within one or more methylation loci (e.g., one or more DMRs). In various embodiments, a methylation biomarker is or includes a change in methylation status that is a change in the number of methylated sites within one or more methylation loci (e.g., one or more DMRs) (e.g., one or more CpG sites). In various embodiments, a methylation biomarker is or includes a change in methylation status that is a change in the frequency of methylation sites within one or more methylation loci (e.g., one or more DMRs). In various embodiments, a methylation biomarker is or includes a change in methylation status that is a change in the pattern of methylation sites within one or more methylation loci (e.g., one or more DMRs).

In various embodiments, methylation status of one or more methylation loci (e.g., one or more DMRs) is expressed as a fraction or percentage of the one or more methylation loci (e.g., the one or more DMRs) present in a sample that are methylated, e.g., as a fraction of the number of individual DNA strands of DNA in a sample that are methylated at one or more particular methylation loci (e.g., one or more particular DMRs). Those of skill in the art will appreciate that, in some instances, the fraction or percentage of methylation can be calculated from the ratio of methylated DMRs to unmethylated DMRs for one or more analyzed DMRs, e.g., within a sample.

In various embodiments, methylation status of one or more methylation loci (e.g., one or more DMRs) is compared to a reference methylation status value and/or to methylation status of the one or more methylation loci (e.g., one or more DMRs) in a reference sample or a group of reference samples. For example, in certain embodiments, the group of reference samples is a plurality of samples obtained from individuals where said samples are known to represent a particular state (e.g., non-pwMS state, certain MS subtype, low severity status, etc.). In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state. Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

In various embodiments, methylation status of one or more methylation loci (e.g., one or more DMRs) is compared to a reference methylation status value and/or to methylation status of the one or more methylation loci (e.g., one or more DMRs) in a reference sample. In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state. Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

In various embodiments, a methylation status of a methylation loci may be based on methylation of one or more reads (e.g., obtained using a NGS technique) mapped to the methylation loci. For example, when analyzing sequencing data obtained from a sequencing technique, e.g., a NGS sequencing technique, e.g., a targeted NGS sequencing technique, sequencing data may include an inferred or probabilistic sequence of base pairs of a DNA fragment. The inferred or probabilistic sequence of base pairs of the DNA fragment is known as a read. The read may be mapped to a methylation loci (e.g., a DMR, a mutation marker) reference sequence, for example, in a genome (e.g., a reference genome, e.g., a reference bisulfite converted genome). Based on a comparison of the read sequence to a reference sequence, individual CpGs or cytosine residues may be identified as being hypermethylated or hypomethylated as compared to a reference state. In certain embodiments, a read-wise methylation value (e.g., a read-wise methylation score) is determined for a read, based pre-determined minimal thresholds that takes into account a number of methylation sites (e.g., CpGs) and a percentage of methylation. For example, in a portion of a sequence read which overlaps a methylation marker (e.g., a DMR), at least 50% (e.g., at least 60%, at least 70%, at least 75%, at least 80%, at least 90%, or all) of CpGs in the overlapping portion being methylated are indicative that a sequence read is hypermethylated.

A sample analyzed using methods and compositions provided herein can be any biological sample and/or any sample including nucleic acids. In various particular embodiments, a sample analyzed using methods and compositions provided herein can be a sample from a mammal. In various particular embodiments, a sample analyzed using methods and compositions provided herein can be a sample from a human subject.

In various instances, a human subject is a subject diagnosed or seeking diagnosis as having, diagnosed as or seeking diagnosis as at risk of having, and/or diagnosed as or seeking diagnosis as at immediate risk of having, MS. In various instances, a human subject is a subject identified as a subject in need of screening/assessment for (e.g., susceptible to) a MS. In certain instances, a human subject is a subject identified as in need of MS screening by a medical practitioner. In various instances, a human subject is identified as in need of MS screening/assessment due to one or more of symptoms, test results, family history, etc. In various instances, a human subject is identified as being high risk and/or in need of screening for MS based on, without limitation, familial history, prior diagnoses, and/or an evaluation by a medical practitioner. In various instances, a human subject is a subject not diagnosed as having, not at risk of having, not at immediate risk of having, not diagnosed as having, and/or not seeking diagnosis for MS.

A sample from a subject, e.g., a human or other mammalian subject, can be a sample of, e.g., blood, blood component (e.g., plasma, buffy coat), cfDNA (cell free DNA), ctDNA (circulating tumor DNA), etc. In various particular embodiments, a sample is a sample of cell-free DNA (cfDNA). cfDNA is typically found in biological fluids (e.g., plasma, serum, or urine) in short, double-stranded fragments. The concentration of cfDNA is typically low, but can significantly increase under particular conditions. cfDNA provides a real-time or nearly real-time metric of the methylation status of a source sample. cfDNA has a half-life in blood of about 2 hours, such that a sample taken at a given time provides a relatively timely reflection of the status of a source sample.

Various methods of isolating nucleic acids from a sample (e.g., of isolating cfDNA from blood or plasma) are known in the art. Nucleic acids can be isolated, e.g., without limitation, standard DNA purification techniques, by direct gene capture (e.g., by clarification of a sample to remove assay-inhibiting agents and capturing a target nucleic acid, if present, from the clarified sample with a capture agent to produce a capture complex, and isolating the capture complex to recover the target nucleic acid).

The amount of DNA (e.g., cfDNA) used in methods described herein is important to accurate determination of methylation status. In some embodiments, from about 8 ng to about 20 ng of DNA is required per sample (e.g., at least Ing, at least 8 ng, at least 10 ng, at least 15 ng, at least 20 ng). In certain embodiments, about 3 to about 4 mL of plasma is required to obtain a sufficient amount of cfDNA for sample processing.

Methylation status can be measured by a variety of methods known in the art and/or by methods provided in this specification. Those of skill in the art will appreciate that a method for measuring methylation status can generally be applied to samples from any source and of any kind, and will further be aware of processing steps available to modify a sample into a form suitable for measurement by a given methodology. Multiple quantitative methylation assays are available. These include COBRA™ which uses methylation-sensitive restriction endonuclease, gel electrophoresis, and detection based on labeled hybridization probes. Another available technique is the Methylation Specific PCR (MSP) for the amplification of DNA segments of interest. This is performed after sodium ‘bisulfite’ conversion of cytosine using methylation-sensitive probes. MethyLight™, a quantitative methylation assay-based, uses fluorescence-based PCR. Another method used is the Quantitative Methylation (QM™) assay, which combines PCR amplification with fluorescent probes designed to bind to putative methylation sites. Ms-SNuPET is a quantitative technique for determining differences in methylation levels in CpG sites. As with other techniques, bisulfite treatment is first performed leading to the conversion of unmethylated cytosine to uracil while methylcytosine is unaffected. PCR primers specific for bisulfite converted DNA are used to amplify the target sequence of interest. The amplified PCR product is isolated and used to quantitate the methylation status of the CpG site of interest.

In certain embodiments, the processing steps involve fragmenting or shearing DNA of the sample. For example, genomic DNA (e.g., gDNA) obtained from a cell, tissue, or other source may require fragmentation prior to sequencing. In certain embodiments, DNA may be fragmented prior to measurement of methylation status using a physical method (e.g., using an ultra-sonicator, a nebulizer technique, hydrodynamic shearing, etc.). In certain embodiments, DNA may be fragmented using an enzymatic method (e.g., using an endonuclease or a transposase). Certain samples, e.g., cfDNA samples, may not require fragmentation. cfDNA fragments are about 100-200 bp in length and may be appropriate for certain methods provided herein. DNA fragments of about 100-1000 bp in length are suitable for analysis in certain NGS techniques described herein including, for example, Illumina® based techniques. Certain technologies may require DNA fragments of about 100-1000 bp range. In contrast, DNA fragments of about 10 kb or longer are suitable for long read sequencing technologies.

Methods of measuring methylation status include, without limitation, methods including whole genome bisulfite sequencing, targeted bisulfite sequencing, targeted enzymatic methylation sequencing, methylation-status-specific polymerase chain reaction (PCR), methods including mass spectrometry, methylation arrays, methods including methylation-specific nucleases, methods including mass-based separation, methods including target-specific capture (e.g., hybrid capture), and methods including methylation-specific oligonucleotide primers. Certain particular assays for methylation utilize a bisulfite reagent (e.g., hydrogen sulfite ions), methylation sensitive restriction enzymes, or enzymatic conversion reagents (e.g., Tet methylcytosine dioxygenase 2, T4 Phage β-glucosyltransferase, and APOBEC).

Bisulfite reagents can include, among other things, bisulfite, disulfite, hydrogen sulfite, sodium metabisulphite, or combinations thereof, which reagents can be useful in distinguishing methylated and unmethylated nucleic acids. Bisulfite interacts differently with cytosine and 5-methylcytosine. In typical bisulfite-based methods, contacting of DNA (e.g., single stranded DNA, double stranded DNA) with bisulfite deaminates (e.g., converts) unmethylated cytosine to uracil, while methylated cytosine remains unaffected. Methylated cytosines, but not unmethylated cytosines, are selectively retained. Thus, in a bisulfite processed sample, uracil residues stand in place of, and thus provide an identifying signal for, unmethylated cytosine residues, while remaining (methylated) cytosine residues thus provide an identifying signal for methylated cytosine residues. Bisulfite processed samples can be analyzed, e.g., by next generation sequencing (NGS) or other methods disclosed herein.

In some embodiments, bisulfite treatment includes subjecting DNA fragments (e.g., double stranded DNA) to one or more denaturation-conversion cycles in order to convert unmethylation cytosines to uracils in the DNA fragments. Denaturation converts double stranded DNA fragments in the sample to single stranded DNA fragments. In some embodiments, bisulfite treatment may be applied prior to library preparation. In some embodiments, bisulfite treatment may be applied after library preparation.

Enzymatic conversion reagents can include Tet methylcytosine dioxygenase 2 (TET2) and T4 Phage β-glucosyltransferase (T4 BGT), among others. TET2 oxidizes 5-methylcytosine and thus protects it from the consecutive deamination by APOBEC. APOBEC deaminates unmethylated cytosine to uracil, while oxidized 5-methylcytosine remains unaffected. Thus, in a TET2 processed sample, uracil residues stand in place of, and thus provide an identifying signal for, unmethylated cytosine residues, while remaining (methylated) cytosine residues thus provide an identifying signal for methylated cytosine residues. TET2 processed samples can be analyzed, e.g., by next generation sequencing (NGS). In certain embodiments, APOBEC refers to a member (or plurality of members) of the Apolipoprotein B mRNA Editing Catalytic Polypeptide-like (APOBEC) family. In certain embodiments, APOBEC may refer to APOBEC-1, APOBEC-2, APOBEC-3A, APOBEC-3B, APOBEC-3C, APOBEC-3D, APOBEC-3E, APOBEC-3F, APOBEC-3G. APOBEC-3H, APOBEC-4, and/or Activation-induced (cytidine) deaminase (AID). In certain embodiments, enzymatic conversion reagents can include a glucosyltransferase (e.g., a β-glucosyltransferase (BGT), e.g., T4-phage β-glucosyltransferase (T4BGT)). T4 BGT transfers the glucose moiety of uridine diphosphoglucose (UDP-glucose) to the 5-hydroxymethylcytosine (5-hmC) residues in double-stranded DNA generating β-glucosyl-5 hydroxymethylcytosine, which aids in locus specific detection of 5-hmC residues and enrichment of 5-hmC containing DNA.

Methods of measuring methylation status can include, without limitation, massively parallel sequencing (e.g., next-generation sequencing) to determine methylation state, e.g., sequencing-by-synthesis, real-time (e.g., single-molecule) sequencing, bead emulsion sequencing, nanopore sequencing, or other sequencing techniques known in the art. In some embodiments, a method of measuring methylation status can include whole-genome sequencing, e.g., measuring whole genome methylation status from bisulfite or enzymatically treated material with base-pair resolution.

In some embodiments, a method of measuring methylation status includes reduced representation bisulfite sequencing e.g., utilizing use of restriction enzymes to measure methylation status of high CpG content regions from bisulfite or enzymatically treated material with base-pair resolution.

In some embodiments, a method of measuring methylation status can include targeted sequencing e.g., measuring methylation status of pre-selected genomic location from bisulfite or enzymatically treated material with base-pair resolution.

In some embodiments, the pre-selection (capture) (e.g., enrichment) of regions of interest (e.g., DMRs) can be done by any suitable method including complementary in vitro synthesized oligonucleotide sequences (e.g., capture baits/probes). Capture probes (e.g., oligonucleotide capture probes, oligonucleotide capture baits) are useful in targeted sequencing (e.g., NGS) techniques to enrich for particular regions of interest in an oligonucleotide (e.g., DNA) sequence. For example, enrichment of target regions is useful when sequences of particular pre-determined regions of DNA are sequenced. In certain embodiments, capture probes are about 10 to 1000 bp long (e.g., about 10 to about 200 bp long) (e.g., about 120 bp long). In certain embodiments, one or more capture probes are targeted to capture a region of interest (e.g., a biomarker herein) corresponding to one or more methylation loci (e.g., methylation loci comprising at least a portion of one or more DMRs, e.g., as found in Supplementary Tables 3, 4, 7, or 8). In certain embodiments, capture probes are targeted to methylation loci that are hypomethylated or hypermethylated. For example, a capture probe may be targeted to particular methylation loci. However, if fragments of DNA corresponding to methylation loci are converted (e.g., bisulfite or enzymatic converted) prior to enrichment using a capture probe, the sequence of the converted DNA fragments will change as described herein due to particular cytosine residues being unmethylated. Therefore, targeting an unconverted DNA region may result in some mismatches if cytosines are hypomethylated. Though capture probe-target sequence hybridization may tolerate some mismatches, a second probe may be required to enrich for DNA regions which are hypomethylated.

In certain embodiments, the capture probes are nucleic acid probes (e.g., DNA probes, RNA probes). In some embodiments, a method may also include identifying mutated regions (e.g., individual nucleotide bases) using targeted sequencing e.g., determining the presence of a mutation in one or more pre-selected genomic locations (e.g., a genomic marker, e.g., a mutation marker). In certain embodiments, mutations may also be identified from bisulfite or enzymatically treated DNA with base-pair resolution.

In some embodiments, a method for measuring methylation status can include Illumina Methylation Assays e.g., measuring over 850,000 methylation sites quantitatively across a genome at single-nucleotide resolution.

Various methylation assay procedures can be used in conjunction with bisulfite treatment to determine methylation status of a target sequence such as a DMR. Such assays can include, among others, Methylation-Specific Restriction Enzyme qPCR, sequencing of bisulfite-treated nucleic acid, PCR (e.g., with sequence-specific amplification), Methylation Specific Nuclease-assisted Minor-allele Enrichment PCR, and Methylation-Sensitive High Resolution Melting. In some embodiments, DMRs are amplified from converted (e.g., bisulfite or enzyme converted) DNA fragments for library preparation.

In some embodiments, a sequencing library may be prepared using converted oligonucleotide fragments (e.g., fragments converted via an enzymatic conversion protocol), wherein the library is prepared, for example, using a TWIST cfDNA library preparation kit protocol, an IDT NGS library preparation protocol, or a modified Illumina or other sequencing platform-compatible library preparation protocol. In some embodiments, the oligonucleotide fragments are DNA fragments which have been converted (e.g., bisulfite or enzyme converted). In certain embodiments, DNA fragments used in preparation of a sequencing library may be single stranded DNA fragments or double stranded DNA fragments. In certain embodiments, a library may be prepared by attaching adapters to DNA fragments. Adapters contain short (e.g., about 100 to about 1000 bp) sequences (e.g., oligonucleotide sequences) that allow oligonucleotide fragments of a library (e.g., a DNA library) to bind to and generate clusters on a flow cell used in, for example, next generation sequencing (NGS). Adapters may be ligated to library fragments prior to NGS. In certain embodiments, a ligase enzyme covalently links the adapter and library fragments. In certain embodiments, adapters are attached to either one or both of the 5′ and 3′ ends of converted DNA fragments.

In certain embodiments, adapters are attached to DNA fragments prior to subjecting DNA to conversion. In certain embodiments, adapters used herein contain a sequence of oligonucleotides that aid in sample identification. For example, in certain embodiments, adapters include a sample index. A sample index is a short sequence (e.g., about 8 to about 10 bases) of nucleic acids (e.g., DNA, RNA) that serve as sample identifiers and allow for, among other things, multiplexing and/or pooling of multiple samples in a single sequencing run and/or on a flow cell (e.g., used in a NGS technique). In certain embodiments, an adapter at a 5′ end, a 3′ end, or both of a converted single stranded DNA fragment includes a sample index. In certain embodiments, an adapter sequence may include a molecular barcode. A molecular barcode may serve as a unique molecular identifier (UMI) to identify a target molecule during, for example, DNA sequencing. In certain embodiments, DNA barcodes may be randomly generated. In certain embodiments, DNA barcodes may be predetermined or predesigned. In certain embodiments, the DNA barcodes are different on each DNA fragment. In certain embodiments, the DNA barcodes may be the same for two single stranded DNA fragments that are not complementary to one another (e.g., in a Watson-Crick pair with each other) in the biological sample. In certain embodiments, DNA fragments may be amplified (e.g., using PCR) after ligation of adapters to DNA fragments. In certain embodiments, at least 40% (e.g., at least at least 50%, at least 60%, at least 70%) of the converted DNA fragments have an adapter attached at both the 5′ and 3′ ends.

In certain embodiments, high-throughput and/or next-generation sequencing (NGS) techniques are used to achieve base-pair level resolution of an oligonucleotide (e.g., a DNA) sequence, permitting analysis of methylation status and/or identification of mutations. For example, in certain embodiments, NGS may include single-end or paired-end sequencing. In single-end sequencing, a technique reads a sequenced fragment in one direction—from one end of a fragment to the opposite end of the fragment. In certain embodiments, this produces a single DNA sequence that then may be aligned to a reference sequence. In paired-end sequencing, a sequenced fragment is read in a first direction from one end of the fragment to the opposite end of the fragment. The sequenced fragment may be read until a specified read length is reached. Then, the sequenced fragment is read in a second direction, which is opposite to the first direction. In certain embodiments, having multiple read pairs may help to improve read alignment and/or identify mutations (e.g., insertions, deletions, inversion, etc.) that may not be detected by single-end reading.

Another method, that can be used for methylation detection includes PCR amplification with methylation-specific oligonucleotide primers (MSP methods), e.g., as applied to bisulfite-treated sample (see, e.g., Herman 1992 Proc. Natl. Acad. Sci. USA 93:9821-9826, which is herein incorporated by reference with respect to methods of determining methylation status). Use of methylation-status-specific oligonucleotide primers for amplification of bisulfite-treated DNA allows differentiation between methylated and unmethylated nucleic acids. Oligonucleotide primer pairs for use in MSP methods include at least one oligonucleotide primer capable of hybridizing with sequence that includes a methylation site, e.g., a CpG site. An oligonucleotide primer that includes a T residue at a position complementary to a cytosine residue will selectively hybridize to templates in which the cytosine was unmethylated prior to bisulfite treatment, while an oligonucleotide primer that includes a G residue at a position complementary to a cytosine residue will selectively hybridize to templates in which the cytosine was methylated cytosine prior to bisulfite treatment. MSP results can be obtained with or without sequencing amplicons, e.g., using gel electrophoresis. MSP (methylation-specific PCR) allows for highly sensitive detection (detection level of 0.1% of the alleles, with full specificity) of locus-specific DNA methylation, using PCR amplification of bisulfite-converted DNA.

Another method that can be used to determine methylation status after bisulfite treatment of a sample is Methylation-Sensitive High Resolution Melting (MS-HRM) PCR (see, e.g., Hussmann 2018 Methods Mol Biol. 1708:551-571, which is herein incorporated by reference with respect to methods of determining methylation status). MS-HRM is an in-tube, PCR-based method to detect methylation levels at specific loci of interest based on hybridization melting. Bisulfite treatment of the DNA prior to performing MS-HRM ensures a different base composition between methylated and unmethylated DNA, which is used to separate the resulting amplicons by high resolution melting. A unique primer design facilitates a high sensitivity of the assays enabling detection of down to 0.1-1% methylated alleles in an unmethylated background. Oligonucleotide primers for MS-HRM assays are designed to be complementary to the methylated allele, and a specific annealing temperature enables these primers to anneal both to the methylated and the unmethylated alleles thereby increasing the sensitivity of the assays.

Another method that can be used to determine methylation status after bisulfite treatment of a sample is Quantitative Multiplex Methylation-Specific PCR (QM-MSP). QM-MSP uses methylation specific primers for sensitive quantification of DNA methylation (see, e.g., Fackler 2018 Methods Mol Biol. 1708:473-496, which is herein incorporated by reference with respect to methods of determining methylation status). QM-MSP is a two-step PCR approach, where in the first step, one pair of gene-specific primers (forward and reverse) amplifies the methylated and unmethylated copies of the same gene simultaneously and in multiplex, in one PCR reaction. This methylation-independent amplification step produces amplicons of up to 109 copies per μL after 36 cycles of PCR. In the second step, the amplicons of the first reaction are quantified with a standard curve using real-time PCR and two independent fluorophores to detect methylated/unmethylated DNA of each gene in the same well (e.g., 6FAM and VIC). One methylated copy is detectable in 100,000 reference gene copies.

Another method that can be used to determine methylation status after bisulfite treatment of a sample is Methylation Specific Nuclease-assisted Minor-allele Enrichment (MS-NaME) (see, e.g., Liu 2017 Nucleic Acids Res. 45 (6): e39, which is herein incorporated by reference with respect to methods of determining methylation status). Ms-NaME is based on selective hybridization of probes to target sequences in the presence of DNA nuclease specific to double-stranded (ds) DNA (DSN), such that hybridization results in regions of double-stranded DNA that are subsequently digested by the DSN. Thus, oligonucleotide probes targeting unmethylated sequences generate local double stranded regions resulting in digestion of unmethylated targets; oligonucleotide probes capable of hybridizing to methylated sequences generate local double-stranded regions that result in digestion of methylated targets, leaving methylated targets intact. Moreover, oligonucleotide probes can direct DSN activity to multiple targets in bisulfite-treated DNA, simultaneously. Subsequent amplification can enrich non-digested sequences. Ms-NaME can be used, either independently or in combination with other techniques provided herein.

Another method that can be used to determine methylation status after bisulfite treatment of a sample is Methylation-sensitive Single Nucleotide Primer Extension (Ms-SNuPE™) (see, e.g., Gonzalgo 2007 Nat Protoc. 2 (8): 1931-6, which is herein incorporated by reference with respect to methods of determining methylation status). In Ms-SNuPE, strand-specific PCR is performed to generate a DNA template for quantitative methylation analysis using Ms-SNuPE. SNuPE is then performed with oligonucleotide(s) designed to hybridize immediately upstream of the CpG site(s) being interrogated. Reaction products can be electrophoresed on polyacrylamide gels for visualization and quantitation by phosphor-image analysis. Amplicons can also carry a directly or indirectly detectable labels such as a fluorescent label, radionuclide, or a detachable molecule fragment or other entity having a mass that can be distinguished by mass spectrometry. Detection may be carried out and/or visualized by means of, e.g., matrix assisted laser desorption/ionization mass spectrometry (MALDI) or using electron spray mass spectrometry (ESI).

Certain methods that can be used to determine methylation status after bisulfite treatment of a sample utilize a first oligonucleotide primer, a second oligonucleotide primer, and an oligonucleotide probe in an amplification-based method. For instance, the oligonucleotide primers and probe can be used in a method of real-time polymerase chain reaction (PCR) or droplet digital PCR (ddPCR). In various instances, the first oligonucleotide primer, the second oligonucleotide primer, and/or the oligonucleotide probe selectively hybridize methylated DNA and/or unmethylated DNA, such that amplification or probe signal indicate methylation status of a sample.

Other bisulfite-based methods for detecting methylation status (e.g., the presence of level of 5-methylcytosine) are disclosed, e.g., in Frommer (1992 Proc Natl Acad Sci USA. 1; 89 (5): 1827-31, which is herein incorporated by reference with respect to methods of determining methylation status).

In certain MSRE-qPCR embodiments, the amount of total DNA is measured in an aliquot of sample in native (e.g., undigested) form using, e.g., real-time PCR or digital PCR.

Various amplification technologies can be used alone or in conjunction with other techniques described herein for detection of methylation status. Those of skill in the art, having reviewed the present specification, will understand how to combine various amplification technologies known in the art and/or described herein together with various other technologies for methylation status determination known in the art and/or provided herein. Amplification technologies include, without limitation, PCR, e.g., quantitative PCR (qPCR), real-time PCR, and/or digital PCR. Those of skill in the art will appreciate that polymerase amplification can multiplex amplification of multiple targets in a single reaction. PCR amplicons are typically 100 to 2000 base pairs in length. In various instances, an amplification technology is sufficient to determine methylations status.

Digital PCR (dPCR) based methods involve dividing and distributing a sample across wells of a plate with 96-, 384-, or more wells, or in individual emulsion droplets (ddPCR) e.g., using a microfluidic device, such that some wells include one or more copies of template and others include no copies of template. Thus, the average number of template molecules per well is less than one prior to amplification. The number of wells in which amplification of template occurs provides a measure of template concentration. If the sample has been contacted with MSRE, the number of wells in which amplification of template occurs provides a measure of the concentration of methylated template.

In various embodiments a fluorescence-based real-time PCR assay, such as MethyLight™, can be used to measure methylation status (see, e.g., Campan 2018 Methods Mol Biol. 1708:497-513, which is herein incorporated by reference with respect to methods of determining methylation status). MethyLight is a quantitative, fluorescence-based, real-time PCR method to sensitively detect and quantify DNA methylation of candidate regions of the genome. MethyLight is uniquely suited for detecting low-frequency methylated DNA regions against a high background of unmethylated DNA, as it combines methylation-specific priming with methylation-specific fluorescent probing. Additionally, MethyLight can be combined with Digital PCR, for the highly sensitive detection of individual methylated molecules, with use in disease detection and screening.

Real-time PCR-based methods for use in determining methylation status typically include a step of generating a standard curve for unmethylated DNA based on analysis of external standards. A standard curve can be constructed from at least two points and can permit comparison of a real-time Ct value for digested DNA and/or a real-time Ct value for undigested DNA to known quantitative standards. In particular instances, sample Ct values can be determined for MSRE-digested and/or undigested samples or sample aliquots, and the genomic equivalents of DNA can be calculated from the standard curve. Ct values of MSRE-digested and undigested DNA can be evaluated to identify amplicons digested (e.g., efficiently digested; e.g., yielding a Ct value of 45). Amplicons not amplified under either digested or undigested conditions can also be identified. Corrected Ct values for amplicons of interest can then be directly compared across conditions to establish relative differences in methylation status between conditions. Alternatively or additionally, delta-difference between the Ct values of digested and undigested DNA can be used to establish relative differences in methylation status between conditions.

In certain particular embodiments, targeted bisulfite or enzymatic sequencing (e.g., using hybrid capture) among other techniques, can be used to determine the methylation status of a methylation biomarker for a disease and/or condition. In certain particular embodiments, targeted bisulfite sequencing, among other techniques, can be used to determine the methylation status of a methylation biomarker that is or includes two or more methylation loci.

Each biomarker CpG (e.g., 1-5804) and DMR (1-1913) herein has been identified as being an indicator of one or more aspects of MS (e.g., presence, risk, subtype, severity, activity, etc.). In some embodiments, at least one or the plurality of biomarkers have been identified by a machine learning technique or by logistic regression. A conclusion is determined regarding MS status if the amount of methylation of one or more biomarkers (e.g., CpGs, DMRs, etc.) differs from the amount of methylation established in control subjects (for the same biomarkers) by a predetermined amount or using a statistical threshold of significance. In some embodiments, the predetermined amount is at least a 10% difference (e.g., 10%, 15%, 20%, 25%, 30%, 35%, 40%, 50%, or more) in the amount of methylation as compared to control subjects (for corresponding biomarkers between target subjects and controls). The percent difference is ((|control−target subject|/control)*100%). In other embodiments, the predetermined amount is at least, 1 percent, 2, percent, 5 percent, 10 percent, 15 percent, 20 percent, 30 percent, 50 percent, 100 percent, or 200 percent difference in the amount of methylation as compared to control subjects (for corresponding biomarkers between target subject and controls). In some embodiments, the predetermined amount is based on statistically significant differences in the amount of methylation as determined by statistical tests and/or statistical significance tests.

The present disclosure describes an abundance of cytosines with significantly altered methylation status between different MS groups (e.g., pwMS vs. healthy controls, PMS vs. RRMS, severe MS vs. non-severe MS, active vs. remitted, etc.)

Those of skill in the art will appreciate that in embodiments in which a plurality of methylation loci (e.g., a plurality of DMRs) are analyzed for methylation status in a method of screening for MS provided herein, methylation status of each methylation locus can be measured or represented in any of a variety of forms, and the methylation statuses of a plurality of methylation loci (preferably each measured and/or represented in a same, similar, or comparable manner) be together or cumulatively analyzed or represented in any of a variety of forms. In various embodiments, the methylation status of each methylation locus can be measured as a methylation portion. In various embodiments, methylation status of each methylation locus can be represented as the percentage value of methylated reads from total sequencing reads compared against reference sample. In various embodiments, methylation status of each methylation locus can be represented as a qualitative comparison to a reference, e.g., by identification of each methylation locus as hypermethylated or hypomethylated.

In some embodiments in which a single methylation locus is analyzed, hypermethylation of the single methylation locus constitutes a diagnosis that a subject is suffering from or possibly suffering from a condition (e.g., MS), while absence of hypermethylation of the single methylation locus constitutes a diagnosis that the subject is likely not suffering from a condition. In some embodiments, hypermethylation of a single methylation locus (e.g., a single DMR) of a plurality of analyzed methylation loci constitutes a diagnosis that a subject is suffering from or possibly suffering from the condition, while the absence of hypermethylation at any methylation locus of a plurality of analyzed methylation loci constitutes a diagnosis that a subject is likely not suffering from the condition. In some embodiments, hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is suffering from or possibly suffering from the condition (e.g., MS), while the absence of hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is not likely suffering from the condition. In some embodiments, hypermethylation of a determined number (e.g., a predetermined number) of methylation loci (e.g., at least 1, 2, 3, 4, 5, 6, 7, 8, 9, 10, 11, 12 or more DMRs) of a plurality of analyzed methylation loci (e.g. 2, 3, 4, 5, 6, 7, 8, 9, 10, 11, 12 or more DMRs) constitutes a diagnosis that a subject is suffering from or possibly suffering from the condition, while the absence of hypermethylation of a determined number (e.g., a predetermined number) of methylation loci (e.g., at least 1, 2, 3, 4, 5, 6, 7, 8, 9, 10, 11, 12, or more DMRs) of a plurality of analyzed methylation loci (e.g., 2, 3, 4, 5, 6, 7, 8, 9, 10, 11, 12 or more DMRs) constitutes a diagnosis that a subject is not likely suffering from the condition.

In some embodiments, methylation status of a plurality of methylation loci (e.g., a plurality of DMRs) is measured qualitatively or quantitatively and the measurement for each of the plurality of methylation loci are combined to provide a diagnosis. In some embodiments, the quantitatively measured methylation status of each of a plurality of methylation loci is individually weighted, and weighted values are combined to provide a single value that can be comparative to a reference in order to provide a diagnosis.

In some embodiments, methylation status may include determination of methylated and/or unmethylated reads mapped to a genomic region (e.g., a DMR). For example, when using particular sequencing technologies as disclosed herein (e.g., NGS, whole genome bisulfite sequencing, etc.), sequence reads are produced. A sequence read is an inferred sequence of base pairs (e.g., a probabilistic sequence) corresponding to all or part of a sequenced oligonucleotide (e.g., DNA) fragment (e.g., cfDNA fragments, gDNA fragments). In certain embodiments, sequence reads may be mapped (e.g., aligned) to a particular, pre-determined region of interest using a reference sequence (e.g., a bisulfite or enzymatic converted reference sequence) in order to determine if there are any alterations or variations in a read. Alterations may include methylation and/or mutations. A region of interest may include one or more genomic markers including a methylation marker (e.g., a DMR), a mutation marker, or other marker as disclosed herein. In certain embodiments, the region of interest at least 100, at least 200, at least 300, at least 400, at least 500, at least 600, at least 700, at least 800, at least 900, at least 1000 base pairs upstream and/or downstream of the at least one of the one or more markers (e.g., DMRs).

For example, in the case of bisulfite or enzymatically treated DNA fragments, treatment converts unmethylated cytosines to uracils, while methylated cytosines are not converted to uracils. Accordingly, a sequence read produced for a DNA fragment that has methylated cytosines will be different from a sequence read produced for the same DNA fragment that does not have methylated cytosine. Methylation at sites where a cytosine nucleotide is followed by a guanine nucleotide (e.g., CpG sites) may be of particular interest.

In certain embodiments, a region of interest may be sequenced to a read depth of at least 100×, at least 200×, at least 300×, at least 400×, at least 500×, at least 600×, at least 700×, at least 800×, at least 900×, at least 1000× or greater. Read depth is the number of times each individual base within a region has been sequenced.

Methods and compositions of the present disclosure can be used in any of a variety of applications. For example, methods and compositions of the present disclosure can be used to screen, or aid in screening for a condition (e.g., MS). In some embodiments, methods and compositions find use in assessment of MS, such as determining whether (or a likelihood that) a subject suffers from MS, determining whether (or a likelihood that) a subject does not suffer from MS, identifying or determining the subtype of MS that a subject suffers from (or likely suffers from), determining a long-term prognosis for a subject, evaluating severity status, determining a treatment course of action, and/or in the treatment of MS. For example, in certain embodiments, any one or more of (i), (ii), (iii), (iv), and (v) may apply: (i) the methods and/or compositions are useful in the diagnosis of MS in a subject; (ii) the methods and/or compositions are useful in subtyping of MS in a subject; (iii) the methods and/or compositions are useful in determining the severity of MS in a subject (iv) the methods and/or compositions are useful in predicting long-term disability progression of MS in a subject; and/or (v) the methods and/or compositions are useful in treating MS in a subject.

In some embodiments, the compositions and methods herein find use with other techniques and compositions for assessing and treating MS in a subject. Screening for MS is not conventionally a single test or discrete event but rather an iterative process that integrates clinical evaluation, neuroimaging, laboratory studies, and longitudinal observation to establish the diagnosis, assess disease severity, and classify disease subtype and activity. A fundamental question in evaluating a patient for MS is whether their symptoms are consistent with a demyelinating disorder of the central nervous system and whether they meet accepted diagnostic criteria. In some embodiments, clinicians conduct history and neurologic examination, focusing on characteristic clinical syndromes such as optic neuritis, partial transverse myelitis, or brainstem and cerebellar syndromes. In some embodiments, to meet MS criteria these episodes must last at least 24 hours and occur in the absence of fever or infection to qualify as MS-related attacks. The neurologic examination may reveal upper motor neuron signs, sensory abnormalities, visual deficits, or ocular motility disturbances, some of which may persist after clinical recovery and provide objective evidence of central nervous system involvement.

In some embodiments, magnetic resonance imaging (MRI) is conducted to demonstrate dissemination in space and dissemination in time, as required by the McDonald diagnostic criteria. Brain and spinal cord MRI, typically performed with gadolinium contrast, allows visualization of demyelinating lesions in characteristic locations, including periventricular, cortical or juxtacortical, infratentorial, and spinal cord regions. Dissemination in space is established by the presence of lesions in at least two of these locations, while dissemination in time can be shown by the simultaneous presence of gadolinium-enhancing (active) and non-enhancing (older) lesions or by the appearance of new lesions on follow-up imaging. Classic radiographic features, such as ovoid periventricular plaques oriented perpendicular to the ventricles (often referred to as Dawson's fingers) and short-segment spinal cord lesions, further support the diagnosis. MRI also serves as an essential tool for excluding alternative diagnoses, such as small vessel ischemic disease or compressive spinal pathology.

Cerebrospinal fluid (CSF) analysis obtained through lumbar puncture is often used as an adjunctive diagnostic tool, particularly when clinical and MRI findings are equivocal. The most important CSF finding in MS is the presence of oligoclonal bands that are unique to the CSF and absent in the serum, reflecting intrathecal immunoglobulin synthesis. An elevated IgG index may also be present. Under the 2017 McDonald criteria, the presence of CSF-specific oligoclonal bands can substitute for evidence of dissemination in time, allowing a diagnosis of MS to be made after a single clinical attack when MRI findings already demonstrate dissemination in space. Evoked potential studies, particularly visual evoked potentials, may be used in selected cases to detect subclinical demyelination, although their role has diminished with the increasing sensitivity of modern MRI techniques.

An essential component of MS screening and diagnosis is the exclusion of disease mimics that can present with similar clinical or radiographic features. Conditions such as neuromyelitis optica spectrum disorder (associated with aquaporin-4 antibodies), myelin oligodendrocyte glycoprotein-associated disease (MOGAD), sarcoidosis, vitamin B12 deficiency, infectious etiologies such as Lyme disease, and systemic inflammatory or vasculitic disorders must be considered and, when appropriate, ruled out through targeted laboratory testing and imaging. Failure to adequately exclude these conditions can result in misdiagnosis and inappropriate treatment.

Once the diagnosis of MS is established or strongly suspected, clinicians turn to assessing disease severity and burden, both at baseline and over time. Clinical disability is most commonly quantified using the Expanded Disability Status Scale (EDSS), which emphasizes ambulation and neurologic function across multiple domains. Although widely used in clinical trials and practice, the EDSS has recognized limitations, particularly its relative insensitivity to cognitive impairment and upper extremity dysfunction. Complementary measures, such as the Multiple Sclerosis Functional Composite, incorporate timed walking tests, manual dexterity assessments, and cognitive evaluation to provide a more multidimensional view of disability. Relapse history, including frequency, severity, and degree of recovery, also provides important prognostic information, as frequent early relapses, incomplete recovery, and early motor or cerebellar involvement are associated with worse long-term outcomes.

MRI also finds use in severity assessment and longitudinal monitoring. Total T2 lesion burden reflects cumulative disease burden, while the presence of new or enlarging lesions and gadolinium-enhancing lesions indicates ongoing inflammatory activity. Measures of brain and spinal cord atrophy, although more technically demanding, are increasingly recognized as markers of neurodegeneration and disease progression and correlate with long-term disability. In parallel, emerging biomarkers such as serum neurofilament light chain levels are gaining attention as indicators of axonal injury and disease activity and may eventually complement imaging and clinical assessments in routine practice.

Subtyping MS is conventionally based primarily on the clinical course over time rather than on a single diagnostic test. Patients may initially present with a clinically isolated syndrome, defined as a first demyelinating event suggestive of MS but not yet meeting full diagnostic criteria. The majority of patients who go on to develop MS initially follow a relapsing-remitting course, characterized by discrete relapses with partial or complete recovery and no steady progression between attacks. Over time, many of these patients transition to secondary progressive MS, in which disability gradually worsens independent of relapses. A smaller subset of patients presents from the outset with primary progressive MS, marked by steady neurologic decline without clear relapses. Modern classification further refines these subtypes by incorporating descriptors of disease activity and progression, such as “active” versus “not active” and “with progression” versus “without progression,” based on clinical relapses, MRI findings, and disability accumulation.

In some embodiments, the methylation biomarkers herein find use with any conventional MS screening/assessment techniques described herein and/or understood in the field. In some embodiments, analysis of the methylation status of the biomarkers herein is conducted in conjunction with other MS screening/assessment techniques such as analysis of quantitative expression values of biomarkers for one or more qualities of MS. In some embodiments, expression is quantified for one or more MS biomarkers selected from NfL, MOG, CD6, CXCL14, CXCL9, CDCP1, CCL20, OPG, IL-12B, APLP1, GH, VCAN, TNFRSF10A, SERPINA9, PRTG, FLRT2, TNFSF13B (BAFF), OPN, CNTN2, and GFAP. In some embodiments, an MS status (e.g., risk, presence, subtype, severity, etc.) is assessed based on the methylation biomarkers, expression biomarkers, and/or any other MS screening/assessment techniques described herein and/or understood in the field.

For any of the embodiments described herein, the methods may comprise treating the subject with an appropriate multiple sclerosis therapy based upon whether the subject is identified as (i) a pwMS, (ii) having BMS, RRMS, PPMS, or SPMS, (iii) disability severity status, (iv) long-term progression trajectory, (v) etc. In some embodiments, the methods comprise treating the subject with a disease-modifying therapy (DMT). In some embodiments, the DMT comprises one or more therapies selected from beta interferon, glatiramer acetate, fingolimod, dimethyl fumarate, diroximel fumarate, teriflunomide, Siponimod, cladribine, a monoclonal antibody, or mitoxantrone. In some embodiments, the monoclonal antibody is selected from, ocrelizumab, natalizumab, and alemtuzumab. In some embodiments, the method comprises treating the subject with one or more therapies selected from stem cell therapy, steroids, beta interferon, cladribine, and Siponimod. In some embodiments, the method further comprises providing to the subject a therapy that is not a DMT described above, but an alternative therapy to relieve one or more symptoms of MS. Suitable alternative therapies include, for example, corticosteroids, plasma exchange, physical therapy, muscle relaxants, medications to reduce fatigue (e.g. amantadine, modafinil, methylphenidate, anti-depressants (e.g. SSRIs), medications to increase walking speed (dalmampridine), medications for pain management, medications to treat sexual dysfunction, insomnia, or bladder and bowel control problems, and the like. In some embodiments, methods comprise selecting an appropriate therapy (e.g., from those above), based on the assessment of a subject described herein.

In various embodiments, a therapeutic agent is administered to a subject prior to and/or subsequent to obtaining the sample from the individual and analyzing the methylation biomarkers described herein. In some embodiments, analysis of the methylation biomarkers described herein guides therapeutic decision making.

In various embodiments the therapeutic agent is a biologic, e.g. a cytokine, antibody, soluble cytokine receptor, anti-sense oligonucleotide, siRNA, etc. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists, e.g. IL-1Ra, IL-1 Trap, sIL-4Ra, etc. Also included are biosimilar or bioequivalent drugs to the active agents set forth herein.

Therapeutic agents for multiple sclerosis include corticosteroids, plasma exchange, ocrelizumab (Ocrevus®), IFN-β (Avonex®, Betaseron®, Rebif®, Extavia®, Plegridy®), Glatiramer acetate (Copaxone®, Glatopa®), anti-VLA4 (Tysabri, natalizumab), dimethyl fumarate (Tecfidera®, Vumerity®), teriflunomide (Aubagio®), monomethyl fumarate (Bafiertam™), ozanimod (Zeposia®), siponimod (Mayzent®), fingolimod (Gilenya®), anti-CD52 antibody (e.g., alemtuzumab (Lemtrada®), mitoxantrone (Novantrone®), methotrexate, cladribine (Mavenclad®, simvastatin, and cyclophosphamide. In addition or alternative to therapeutic agents, other treatments for multiple sclerosis include lifestyle changes such as physical therapy or a change in diet. The methods herein also provide for combination therapy of one or more therapeutic agents and/or additional treatments, where the combination can provide for additive or synergistic benefits.

Pharmaceutical compositions may be administered for prophylactic (e.g., before diagnosis of a patient with multiple sclerosis) or for treatment (e.g., after diagnosis of a patient with multiple sclerosis) purposes. Preventing, prophylaxis or prevention of a disease or disorder as used in the context of this invention refers to the administration of a composition to prevent the occurrence or onset of multiple sclerosis or some or all of the symptoms of multiple sclerosis or to lessen the likelihood of the onset of a disease or disorder. Treating, treatment, or therapy of multiple sclerosis shall mean slowing, stopping or reversing the disease's progression by administration of treatment according to the present invention.

### EXPERIMENTAL

### Materials and Methods

### Clinic Cohort and Sample Collection.

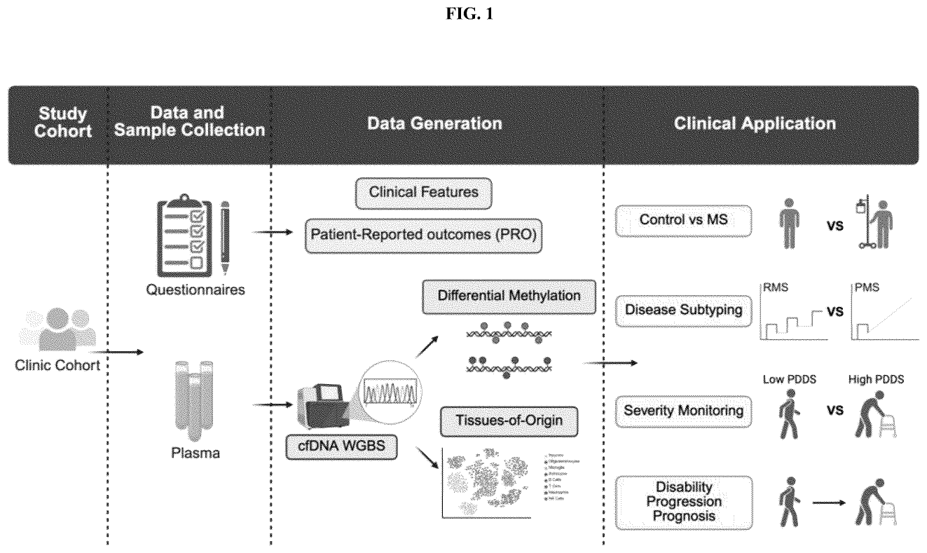

Participants enrolled in a clinic-based long-term natural history cohort study (Prospective Investigation of Multiple Sclerosis in the Three Rivers Region [PROMOTE], Pittsburgh) donated samples during 2017-2021. The current study (FIG. 1; Supplementary Table 1) included 57 samples from adults (≥18 years of age) with a neurologist-confirmed diagnosis of MS according to the 2017 McDonald criteria and 18 samples from adults without MS as controls. Clinical and demographic data were obtained from electronic health records and the cohort registry. MS subtype and relapse status were determined by the treating neurologists. An acute relapse could be a clinical and/or radiologic event within 3 months of sample collection. Clinical relapses were defined as having new or recurrent neurological symptoms (deemed MS-relevant by clinicians) lasting persistently ≥24 hours without fever or infection (≥30 days from the onset of a preceding event). A radiological relapse was defined as having either a new T1-enhancing lesion and/or a new or enlarging T2-fluid-attenuated inversion recovery (FLAIR) hyperintense lesion based on clinical radiology reports of routine brain, orbit, or spinal cord magnetic resonance imaging studies. Participants completed patient-reported outcomes (PROs) that have already been clinically implemented in local practice, including Patient Determined Disease Steps (PDDS), via electronic or paper questionnaires. Research venous blood samples were collected during clinic visits. Plasma samples were isolated within four hours of phlebotomy by centrifugation first at 2,000 g for 10 minutes, and then at 15,000 g for 10 minutes at room temperature, and stored at −80° C. until cfDNA extraction and sequencing.

Whole-Genome Bisulfite Sequencing of cfDNA.

cfDNA was extracted from 2 mL plasma using the MagMAX™ Cell-Free DNA Isolation Kit (Applied Biosystems A29319) following the manufacturer's protocol. The concentration and size distribution of cfDNA were measured using Qubit 1×dsDNA High Sensitivity kit (Invitrogen) and BioAnalyzer (Agilent). Five ng cfDNA underwent bisulfite conversion by the EZ-96 DNA Methylation-Gold Kit (Zymo D5008). Subsequently, cfDNA methylation libraries were prepared using the Accel-NGS® Methyl-Seq DNA Library Kit (Swift 30096) and Methyl-Seq Unique Dual Indexing Kit (SWIFT 390384). To minimize batch effects, all cfDNA samples were processed in a single batch for both bisulfite conversion and library preparation. Qualified libraries were pooled and sequenced on the Illumina NovaSeq S6000 PE150 platform with a 20% spike of PhiX.

### Pre-Process of Whole-Genome Bisulfite Sequencing Data.

Paired-end WGBS from cfDNA was pre-processed using an internal pipeline. Based on FastQC results the distribution of four nucleotides along the sequencing cycle, the adapter was trimmed by Trim Galore! (v0.6.0)⁵⁵ with cutadapt (v2.1.0)56 and with parameters “--clip_R1 8--clip_R2 15 --three_prime_clip_R1 5 --three_prime_clip_R2 5”. After the adapter trimming, reads were aligned to the human genome (GRCh37, human_glk_v37.fa) by Biscuit (v0.3.16.20200420) with default parameters. PCR-duplicate reads were marked by samblaster.⁵⁸ Only high-quality reads were used for downstream analyses (uniquely mapped, properly paired, mapping quality score of 30 or greater, and not a PCR duplicate). The methylation level at each CpG was called by Bis-SNP (v0.90)⁵⁹ using default parameters in bissnp_easy_usage.pl. Only CpGs at autosomes were used for downstream analyses.

Identification of Differentially Methylated CpGs and Differentially Methylated Regions from WGBS Data.

A beta-binomial model implemented in RADMeth³⁸ was used to calculate DMRs between MS and controls, between different MS subtypes, and between pwMS with high and low PDDS scores, adjusting for sex as a covariate. Command adjust used “-bins 1:100:1” as parameters. To merge adjacent differentially methylated CpGs, dmrs with -p 0.1 were used. The threshold to filter DMRs were q value <0.1 and absolute methylation difference >0.1.

Visualization of Differentially Methylated Regions from cfDNA Whole-Genome Bisulfite Sequencing Data.

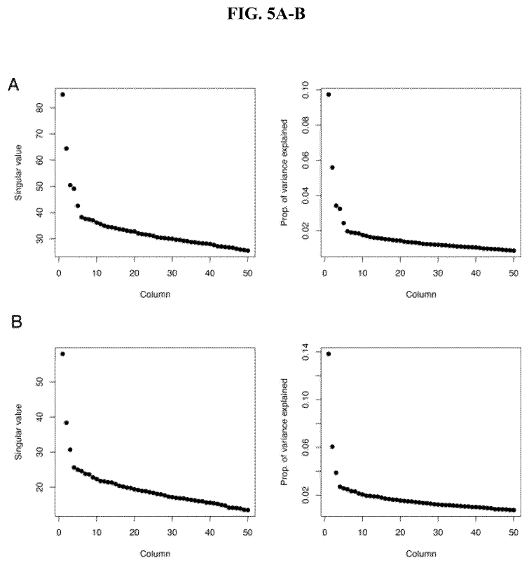

DMRs from the 75 study samples were merged into a single location file and the DNA methylation levels were calculated at these DMRs from all samples. Only DMRs with <80% missing data were retained for the analysis. The missing data was imputed using the impute.knn function in the impute package (v1.70.0) in R (v4.2.0) with parameters of “maxp=“p”, rowmax=0.8”. After scale transformation, the svd function was applied in R to the methylation matrix. The first 50 singular values were plotted against the proportion of variance explained and the elbow point was chosed. Based on the absolute and proportion of variance explained by the top SVDs (FIG. 5), the top six singular value decomposition components (SVDs) were chosen for DMRs between different MS subtypes, and the top four SVDs for DMRs when comparing between pwMS with high vs low PDDS scores. Then, used umap (v0.2.10.0) was used in R for dimension reduction and visualization.

### Enrichment Analysis of Differentially Methylated Regions.

The 15-state chromHMM result at GM12878 from the Epigenome Roadmap was used to compute the enrichment or depletion of DMRs. The p-value was calculated against random intervals with the same chromosome and sizes using the Fisher exact test after multiple test correction (via Benjamini-Hochberg). DMRs were lifted up to the hg38 genome by liftOver.⁶⁰ Gene Ontology (GO) and motif enrichment were computed by g:profile.⁶¹

Tissue-of-Origin Analysis Using cfDNA Whole-Genome Bisulfite Sequencing Data.

Only high-quality reads mapped to the cell type-specific marker regions were used for the tissue-of-origin analysis. After downloading cell type-specific DNA methylation markers (Atlas. U25.14.hg19.tsv, raw.githubusercontent.com/nloyfer/UXM_deconv/main/supplemental/), the bam files were converted to the pat format using wgbstools (v0.2.2) bam2pat with default parameters the tissue-of-origin fraction in each sample was estimated using uxm deconv with default parameters.³⁵

### Whole-Genome Bisulfite Sequencing Coverage Simulation for Assessing Tissue-of-Origin Accuracy.

The processed bam files were merged from non-MS control samples and low-quality reads were filtered, resulting in ~20× effective coverage representing the population average. The merged bam files were randomly sampled at different genomic coverages ranging from 0.01× to 12× the tissue-of-origin fraction was estimated as mentioned above at each step. This process was repeated 100 times to build a distribution and estimated variance around the sample mean at different coverages.

Linear Mixed-Effects Model for Examining the Interaction Effect Between Baseline cfDNA Methylation Level and Time on Disability Severity Trajectory.

To evaluate the association between baseline biomarker (cfDNA methylation) levels and disability progression over time, a linear mixed-effects analysis was conducted using the Imer function from the lme4 package (v1.1)⁶² in R (v4.2.0). The outcome variable was repeated measures of patient-reported disability severity (e.g., PDDS scores), collected at multiple time points for each patient after sample collection for baseline cfDNA methylation profiling. Fixed effects included: (1) time (as a continuous variable), (2) baseline biomarker group (categorized as “High” vs “Low” based on median cfDNA methylation levels in specific genomic regions), and (3) an interaction term (time×biomarker group) to assess potential differences in disability severity trajectories between groups. Age (continuous), sex, race, disease duration, DMT type at sample collection, DMT effectiveness at sample collection, and follow-up duration (time from sample collection to outcome assessment) were controlled as covariates. (This study population included only one ethnicity group, non-Hispanic). A random intercept for each participant accounted for repeated within-person observations.

The model specification is as follows:

Severityy=β₀+β₁·Timeᵢⱼ+β₂·Groupᵢ+β₃·(Timeᵢⱼ×Groupᵢ)+β₄·Ageᵢ+β₅·Sexᵢ+β₆·Raceᵢ+β₇·DiseaseDurationᵢ+β₈·DMTtypeᵢ+β₉·DMTeffectiveᵢ+β₁₀·FollowUpᵢ+μᵢ+εᵢⱼ

### Where:

Severityij: Disability severity for patient i at time j.
β0: Fixed intercept.
β1: Fixed effect of time.
β2: Fixed effect of biomarker group (cfDNA methylation level).
β3: Interaction effect between time and biomarker group.
β4, β5, and β6 represent the effects of age, sex, and race, respectively.
β7: Disease duration time.
β8: DMT type at sample collection.
β9: DMT effectiveness at sample collection.
β10: Follow-up duration time.
ui: Random intercept for patient i.
ϵij: Residual error term.
   The ImerTest package (v3.1)₆₃ was used to obtain p-values for the fixed effects using Satterthwaite's method for approximating degrees of freedom. Model assumptions were checked by examining residual plots for homoscedasticity and normality, statistical significance was set at a two-sided alpha level of 0.01, and multiple comparisons were corrected for using the Bonferroni procedure when testing multiple biomarkers to control the false discovery rate.

### PDDS Interpolation.

Given the pragmatic study design of the clinic-based cohort, PDDS scores were not always collected at identical time intervals for all participants. To address this, linear interpolation was performed to facilitate the analysis of baseline cfDNA methylation profiles in relation to longitudinal trajectories of PDDS scores. Using the intrepid function from SciPy (v1.12.0),⁶⁴ each participant's scores were interpolated at 50-day intervals, covering the period from day 0 to day 2,000 after blood sample collection.

### Candidate Genomic Regions for the Linear Mixed Model.

Genomic regions without missing data between pwMS with higher PDDS (PDDS≥4) and lower PDDS score (PDDS<4) at baseline were selected. Next, regions located within 300 bp of one another were merged using bedtools,⁶⁵ resulting in 2,316,371 total regions. Samples containing >90% missing values across these regions were excluded in subsequent analyses.

### Methylation-Based Prognostic Risk Score and Progression-Free Survival Analysis.

Prognostic regions from the linear mixed-effects model at a false discovery rate of <0.01 were first selected. Next, regions were ranked by the product of their absolute value of estimated effect size (time×biomarker interaction term) and −log₁₀(FDR) (time×biomarker interaction term) and retained the top 100 informative regions. For each participant, the methylation-based prognostic risk score (MBPRS) was calculated as the weighted sum of baseline cfDNA methylation at these regions, using the corresponding effect sizes (time×biomarker interaction term) as weights. Missing methylation values were imputed via the impute.knn function (impute v1.70.0) in R (v4.2.0) with default settings.

Progression-free survival (PFS) was analyzed using the survival package (v3.8) in R (v4.2.0). The event time was defined as the first visit with PDDS≥4 (i.e., requiring ambulatory assistance). Patients were ten dichotomized into “High” vs “Low” MBPRS groups based on the median score of baseline cfDNA methylation level of the prognostic regions and group differences in PFS were assessed by the log-rank test. To validate MBPRS specificity, 1,000 sets of matched random genomic intervals (matched chromosomes and region sizes) were generated. For each set, baseline methylation levels were extracted, the original weights were compared to compute the control MBPRS scores, the same log-rank test was performed. From the resulting distribution of permutation p-values, an empirical p-value was derived for the observed separation.

### Machine Learning Models.

For all machine learning methods described below, seven commonly used machine learning models were evaluated and the best performing model was reported. Models were: LogisticRegression via “l2” or “elasticnet”, support vector machine (SVM) via “rbf” or “linear” kernels, XGBoost, GradientBoostingClassifier, and LightGBM via LGBMClassifier. Parameter tuning was conducted within each model. For DMR features, various transformations (e.g., raw input, top 2-20 SVD components, recursive feature elimination) were tested.

### Machine Learning Models for Comparing MS Vs Non-MS Controls.

A 10-fold cross-validation scheme was employed. In each fold, 90% of the MS cases and 90% of the controls constituted the training set, while the remaining 10% served as the held-out test set. This approach ensured balanced case vs control ratios in both training and test sets. Using a beta-binomial model (RADMeth), differentially methylated CpGs were identified and adjacent sites were merged to form DMRs (used dmrs in RadMeth with −p 0.1). Methylation densities were computed for each DMR. Missing data were marked as NA and processed using NumPy's nan_to_num function. Singular value decomposition (SVD) was performed and the top two SVD components were selected as input features based on the proportion of variance explained. A logistic regression model (l2 LogisticRegression from scikit-learn, v1.1.1)⁶⁶ was trained with the following parameters: “penalty=‘l2’, dual=False, tol=1e-5, C=120, fit_intercept=True, intercept_scaling=1, class_weight=‘balanced’, solver=‘lbfgs’, max_iter=1000”. To calculate all possible differential tissue-of-origin patterns in CNS and immune cells, a one-sided Mann-Whitney U test was conducted to identify cell types (within CNS and immune cell types) that differed significantly (p<0.05) in group comparisons. Cell types meeting this criterion in any comparison were included as features. A gradient boosting classifier (GradientBoostingClassifier from scikit-learn) was trained with the following parameters: “n_estimators=200, learning_rate=0.2, max_depth=10, min_samples_split=2, min_samples_leaf=1, subsample=0.8, max_features=‘log 2’”. Because each participant had only one historical blood NfL or GFAP value, a simple logistic regression model was applied (C=1.0, using StratifiedKFold for the 10-fold cross-validation).

### Machine Learning Models for Comparing PMS Vs RRMS.

The same 10-fold cross-validation scheme and preprocessing steps (as in the MS vs controls comparison) was employed. For DMRs features, instead of taking the top SVD components, recursive feature elimination was employed with cross-validation (RFECV from scikit-learn) in each fold to refine the final features (“step=1,scoring=‘accuracy’,min_features_to_select=1”). A LightGBM classifier (LGBMClassifier in lightgbm) was trained with the following parameters: “n_estimators=100, learning_rate=0.1, num_leaves=31, max_depth=1, reg_alpha=0.1, reg_lambda=0.1, subsample=0.8, colsample_bytree=0.8, min_child_samples=10”. For differential tissue-of-origin features, given the smaller sample size of PMS relative to RRMS, the tissue-of-origin p-value cutoff was relaxed to 0.1. A one-sided Mann-Whitney U test (p<0.1) was performed to select significantly altered cell types. A SVM was trained with the following parameters: “C=8.0, kernel=‘rbf’, probability=True”.

### Machine Learning Models for Comparing Actively Relapsing RRMS Vs Stable Remission RRMS.

For DMR features, the top three SVD components (based on the proportion of variance explained) of DMR methylation levels were retained as input. An SVM model was trained with the following parameters: “kernel=‘rbf’, probability=True, gamma=‘scale’, C=0.1”. For differential tissue-of-origin features, given the smaller sample size of actively relapsing RRMS relative to stable remission RRMS, the p-value cutoff was relaxed to 0.2. A logistic regression model with the following parameters was then used: “penalty=‘l2’, dual=False, tol=0.0001, C=100, fit_intercept=True, intercept_scaling=1, class_weight=‘balanced’, solver=‘lbfgs’, max_iter=100”

### Machine Learning Models to Distinguish Different Disability Severity Levels.

Samples lacking PDDS scores were excluded, retaining 67. For exploring given the greater phenotypic distinction, samples with PDDS≤1 (least severe, mild or normal) or ≥6 (most severe) were used, thereby reducing the sample size to 46. The same 10-fold cross-validation and preprocessing strategies (as in MS vs control comparisons) was applied. For DMRs features, XGBoost⁶⁷ was used with the following parameters: “max_depth=3, learning_rate=0.4, n_estimators=1000, class_weight=‘balanced’, objective=‘binary:logistic’, subsample=0.667”. For differential tissue-of-origin features, all immune and epithelial cell types were tested using a p-value cutoff of 0.1, and a GradientBoostingClassifier was trained with the following parameters: “n_estimators=200, learning_rate=0.15, max_depth=2, min_samples_split=2, min_samples_leaf=2, subsample=0.8, max_features=‘log 2’”.

### Machine Learning Approach to Predict the Future Disease Severity.

Only samples with PDDS score measurements at ≥3 different time points were included. The 5,392 potentially “prognostic regions” identified by the linear mixed-effects model were the focus. At ~1,000 days post-blood draw, samples with PDDS≤1 or ≥6 were retained for analysis. A RepeatedStratifiedKFold (five folds, repeated 100 times) was used. The GradientBoostingClassifier was trained with the following parameters: “n_estimators=100, learning_rate=0.1, max_depth=5, min_samples_split=2, min_samples_leaf=1, subsample=0.8, max features=‘log 2’”.

### Example 1

### Differentially Methylated CpGs and Regions Separate MS Vs Controls and Across MS Subtypes

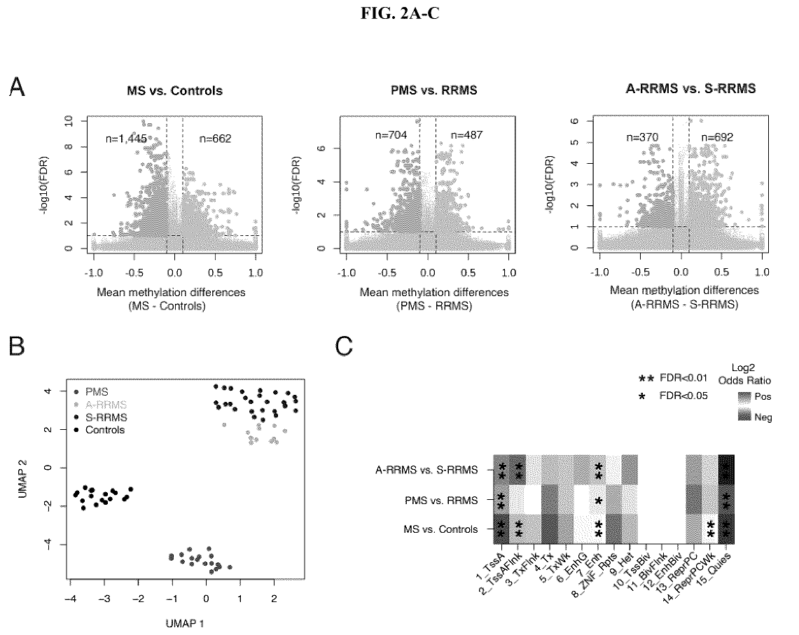

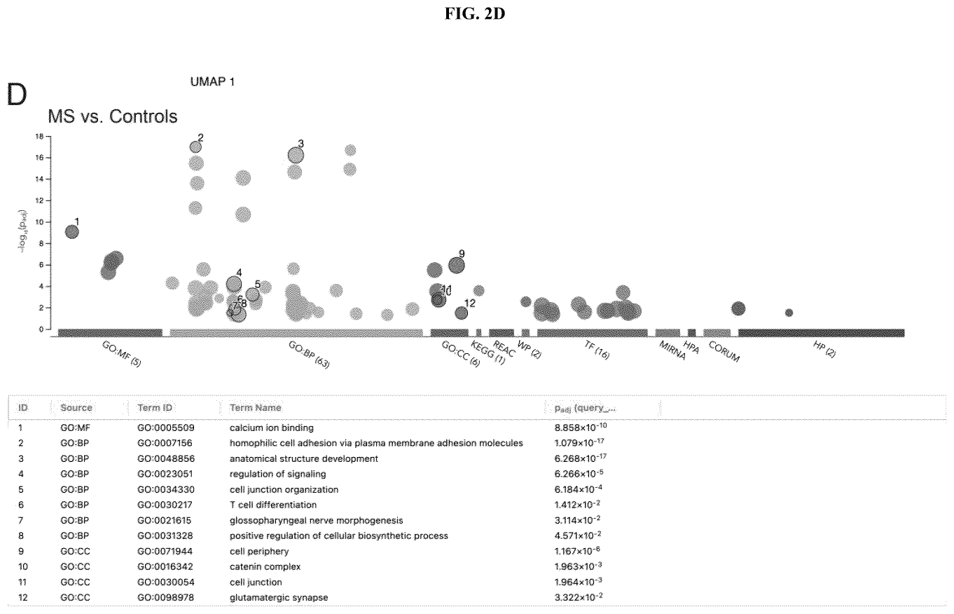

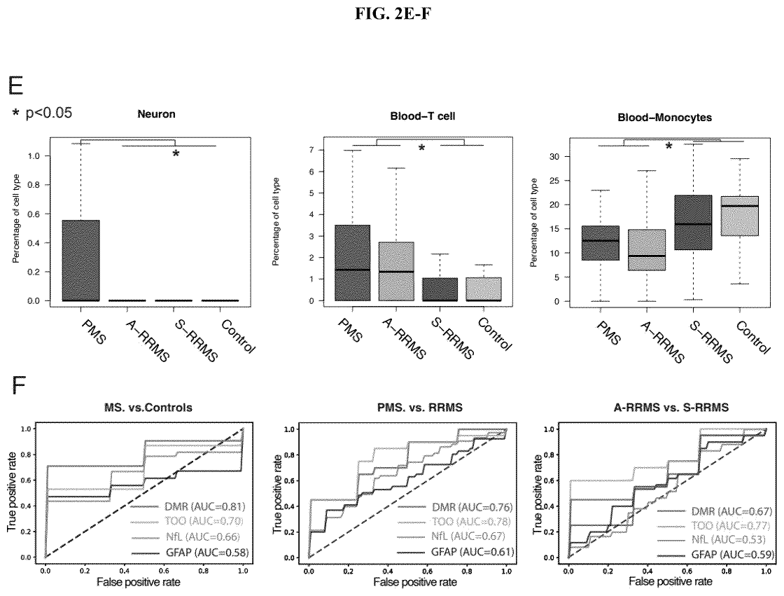

Two mL of plasma from 57 pwMS and 18 non-MS controls enrolled in a clinic-based prospective cohort (PROMOTE) was collected (Table 1). Among the MS samples, 12 were obtained within 3 months of an active relapse from RRMS, 27 during stable remission from RRMS, and 18 from PMS (112 PPMS or SPMS) (FIG. 1). cfDNA WGBS data was generated with a median of ~56 million paired-end reads per sample. After removing low-quality reads, a median of ~34 million paired-end reads per sample was retained, covering ~22 million CpGs at 1.5× per CpG (Supplementary Table 2). Using a beta-binomial regression model (RADMeth³⁸), significant DNA methylation changes between pwMS and controls as well as across MS subtypes was identified (FIG. 2A). Specifically, 662 hypermethylated and 1,445 hypomethylated CpGs were detected in pwMS compared to controls (FDR<0.1, absolute methylation difference >0.1). Applying the same model and thresholds, 487 hypermethylated and 704 hypomethylated CpGs were identified in PMS relative to RRMS. Similarly, 692 hypermethylated and 370 hypomethylated CpGs were observed in active-relapsing RRMS (A-RRMS) compared to stable-remission RRMS (S-RRMS) (Supplementary Table 3). Adjacent differentially methylated CpGs were merged into differentially methylated regions (DMRs) using RADMeth. This yielded 672 DMRs when comparing pwMS vs controls, 342 DMRs when comparing PMS vs RRMS, and 387 DMRs when comparing A-RRMS vs S-RRMS (Supplementary Table 4). Due to the inherent noise in methylation measurements from low-coverage WGBS, a strategy previously developed for single-cell epigenetic analyses was used. Singular value decomposition (SVD)⁴¹ was performed on the DNA methylation matrix for DMRs and the top components that explained the largest fraction of variability were selected (FIG. 5A). Uniform Manifold Approximation and Projection (UMAP)⁴² was applied for dimensional reduction, revealing distinct separations between MS and controls and among the MS subtypes (FIG. 2B).

TABLE 1
Participant Profiles.

| | Control | | A-RRMS | | S-RRMS | | PMS | | Total | | |
|---|---|---|---|---|---|---|---|---|---|---|---|
| | (N = 18) | | (N = 12) | | (N = 27) | | (N = 18) | | (N = 75) | | p value |
| Age at Sample Collection | 57.3 | (11.0) | 41.5 | (9.5) | 48.3 | (11.6) | 61.1 | (7.7) | 52.5 | (12.3) | 0.053 |
| [Y, Mean (SD)] | | | | | | | | | | | |
| Disease Duration | NA | | 14.5 | (11.4) | 19.7 | (14.2) | 27.5 | (12.3) | 21.1 | (13.8) | |
| [Y, Mean (SD)] | | | | | | | | | | | |
| Sex [N (%)] | | | | | | | | | | | 0.017 |
| Female | 9 | (50.0%) | 7 | (58.3%) | 23 | (85.2%) | 15 | (83.3%) | 54 | (72.0%) | |
| Male | 9 | (50.0%) | 5 | (41.7%) | 4 | (14.8%) | 3 | (16.7%) | 21 | (28.0%) | |
| Race [N (%)] | | | | | | | | | | | 0.522 |
| Asian | 0 | (0.0%) | 0 | (0.0%) | 1 | (3.7%) | 0 | (0.0%) | 1 | (1.3%) | |
| Black | 1 | (5.6%) | 3 | (25.0%) | 2 | (7.4%) | 3 | (16.7%) | 9 | (12.0%) | |
| White | 17 | (94.4%) | 9 | (75.0%) | 24 | (88.9%) | 15 | (83.3%) | 65 | (86.7%) | |
| Ethnicity [N (%)] | | | | | | | | | | | NA |
| Not Hispanic or Latino | 18 | (100.0%) | 12 | (100.0%) | 27 | (100.0%) | 18 | (100.0%) | 75 | (100.0%) | |
| PDDS [N (%)] | | | | | | | | | | | <0.001 |
| PDDS ≤1 | 13 | (72.2%) | 8 | (66.7%) | 11 | (40.7%) | 1 | (5.6%) | 33 | (44.0%) | |
| PDDS <4 | 13 | (72.2%) | 9 | (75.0%) | 17 | (63.0%) | 5 | (27.8%) | 44 | (58.7%) | |
| PDDS ≥4 | 1 | (5.6%) | 3 | (25.0%) | 9 | (33.3%) | 11 | (61.1%) | 24 | (32.0%) | |
| PDDS ≥6 | 1 | (5.6%) | 2 | (16.7%) | 3 | (11.1%) | 7 | (38.9%) | 13 | (17.3%) | |

### Example 2

DMRs Across MS Subtypes are Associated with Neuronal and Immune-Related Gene Regulatory Processes

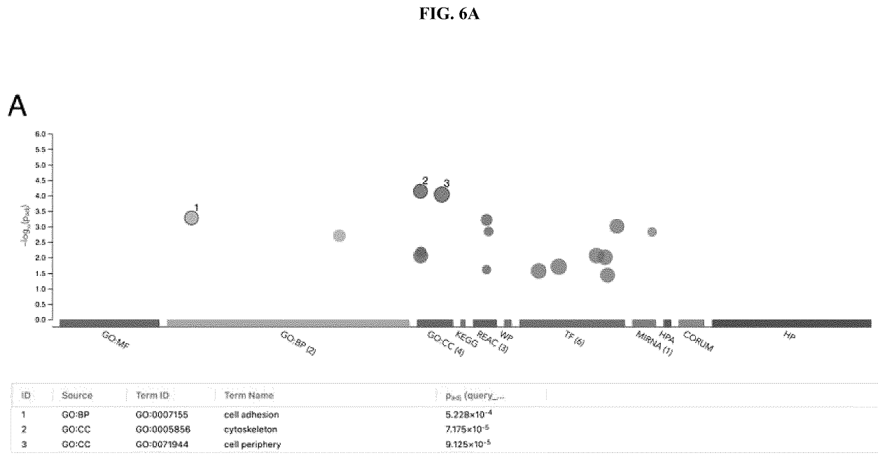

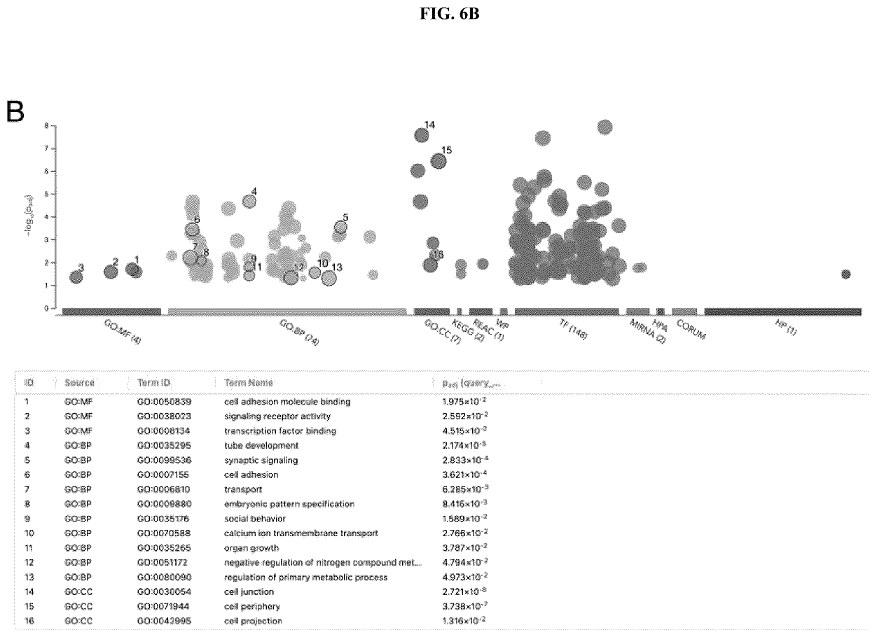

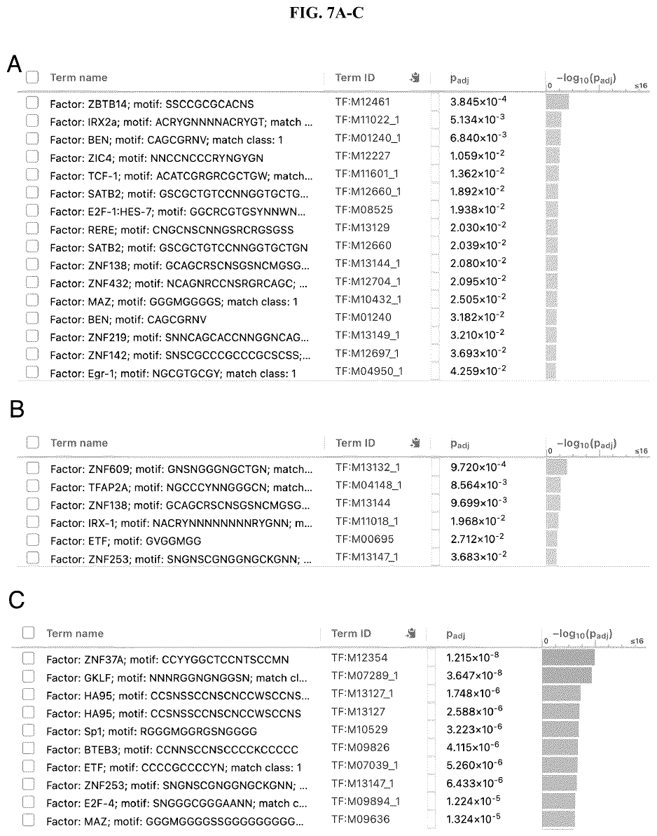

To assess the gene regulatory potential of these DMRs, chromHMM states characterized in immune cells were leveraged. All identified DMRs showed significant enrichment in active promoter and enhancer states and depletion in quiescent states (FIG. 2C, FDR<0.05, Fisher Exact test). DMRs differentiating MS from controls and DMRs distinguishing A-RRMS from S-RRMS were enriched at transcription start site (TSS) flanking states (FDR<0.01, Fisher Exact test), while DMRs differentiating MS and controls further showed significant depletion in weak Polycomb repressive states (FDR<0.01, Fisher Exact test). In Gene Ontology analysis, these DMRs were highly enriched in both neuronal and immune-related processes (FIG. 2D, FIG. 6; Supplementary Table 5). For instance, DMRs distinguishing MS from controls were enriched in T cell differentiation, calcium ion binding, cell adhesion, and anatomical structure development, whereas DMRs separating A-RRMS and S-RRMS were enriched in synaptic signaling. Motif analysis further revealed multiple transcription factor binding sites (TFBSs) significantly overrepresented in all DMRs (FIG. 7). For example, ZBTB14, a regulator of monocyte and macrophage development,⁴⁵ was the most enriched TFBS in DMRs, distinguishing between MS and controls. ZNF609, a modulator of myoblast proliferation, was the most enriched TFBS in DMRs, distinguishing between PMS and RRMS. Finally, ZNF37A, previously implicated in myotonic dystrophy, was the most enriched TFBS in DMRs, distinguishing A-RRMS from S-RRMS.

### Example 3

Tissue-of-Origin Obtained from cfDNA Methylation Varied Significantly Across MS Subtypes

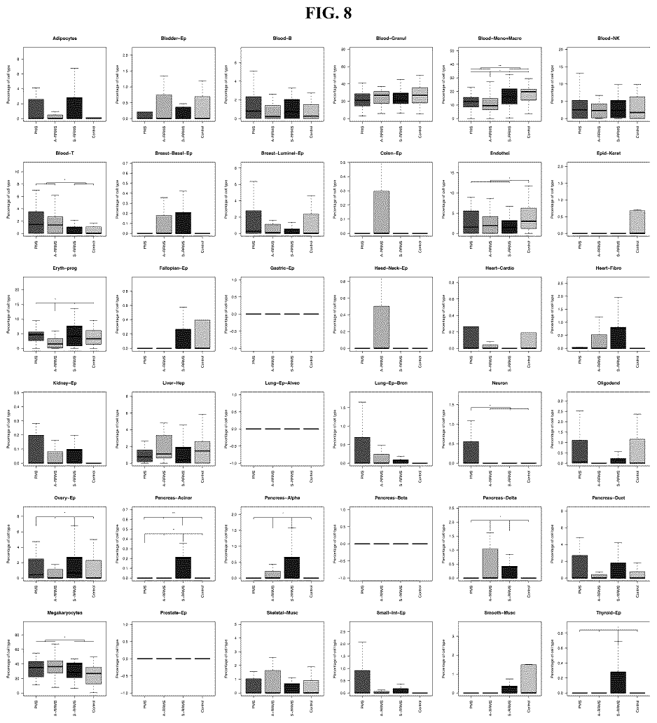

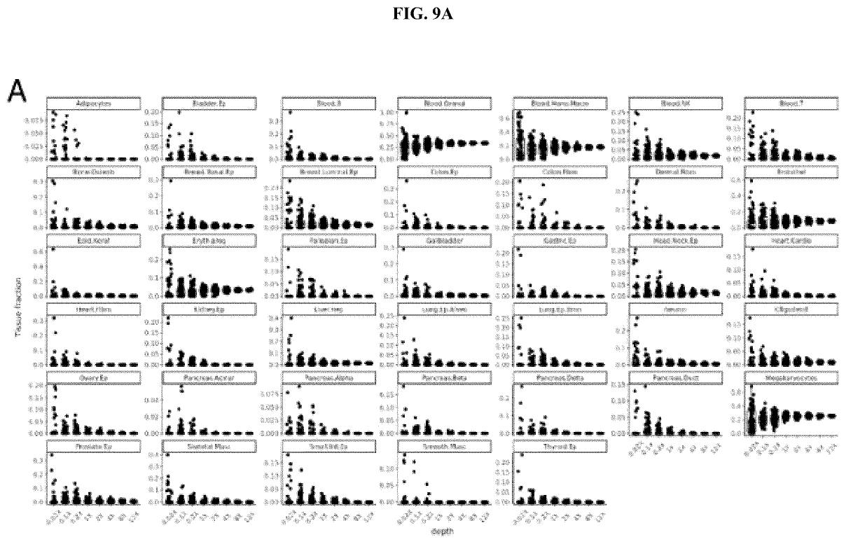

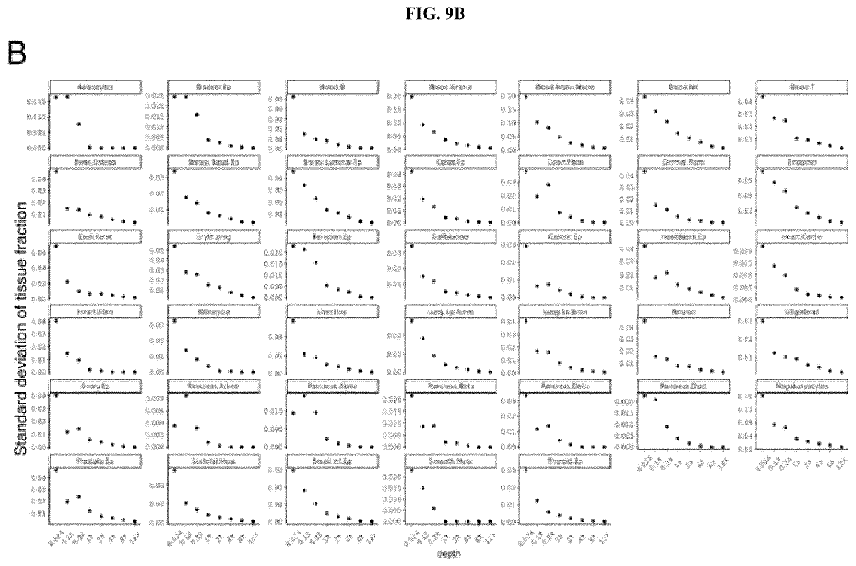

The tissue-of-origin of cfDNA molecules can be identified through their DNA methylation profiles. By deconvoluting cfDNA methylation haplotypes, significantly increased neuronal cell death was observed in PMS compared to other subgroups, and elevated T cell death weas observed in both PMS and A-RRMS relative to S-RRMS and controls (FIG. 2E, FIG. 8; Supplementary Table 6). These findings align with previous reports highlighting neuronal cell death in PMS and T cell-mediated pathogenesis in MS.⁴⁸ Significantly lower monocyte cell death and higher megakaryocyte cell death was detected in PMS and A-RRMS, along with reduced erythroid progenitor cell death specifically in the A-RRMS group (FIG. 8, one-side Mann-Whitney U test, p<0.05). While MS pathogenesis research has investigated T cell-mediated mechanisms, evidence also supports the involvement of other cell types, including peripheral monocytes⁴⁹ and megakaryocytes. Based on simulation analysis, higher coverage (≥8×) WGBS data is used to validate these observations (FIG. 9).

### Example 4

cfDNA Methylation Level and its Tissue-of-Origin Informed MS Diagnosis and Subtype Classification

It was examined whether DMR-based cfDNA methylation levels or tissue-of-origin patterns could distinguish MS from controls and among MS subtypes (FIG. 2F). Using 10-fold cross-validations, DMRs and significantly differentiated tissue-of-origin patterns (in CNS and immune cells) were identified within a training set. Machine-learning models were trained on these features and their performance evaluated in the held-out test set. For distinguishing MS from controls, DMRs alone achieved an area under the curve (AUC) of 0.81 (standard deviation [SD] 0.17), while tissue-of-origin features alone yielded AUC 0.70 (SD 0.19). DMRs and tissue-of-origin features outperformed neurofilament light chain (NfL: AUC 0.66) or glial fibrillary acidic protein (GFAP: AUC 0.58) measured in a previous study from the same clinic-based cohort as historical comparison in a subset of the current study.⁵¹ For distinguishing PMS from RRMS, DMRs achieved AUC 0.76 (SD 0.20) while tissue-of-origin features achieved AUC 0.78 (SD 0.21), outperforming NfL (AUC 0.67) and GFAP (AUC 0.61) in historical comparisons. For distinguishing A-RRMS from S-RRMS, DMRs achieved AUCs 0.67 (SD 0.29) while tissue-of-origin features achieved AUC 0.77 (SD 0.26), outperforming NfL (AUC 0.53) and GFAP (AUC 0.59) in historical comparisons. These findings support that cfDNA methylation and its tissue-of-origin may serve as biomarkers for MS diagnosis and subtype classification.

### Example 5

cfDNA Methylation Level and Tissue-of-Origin Distinguished Disability Severity.

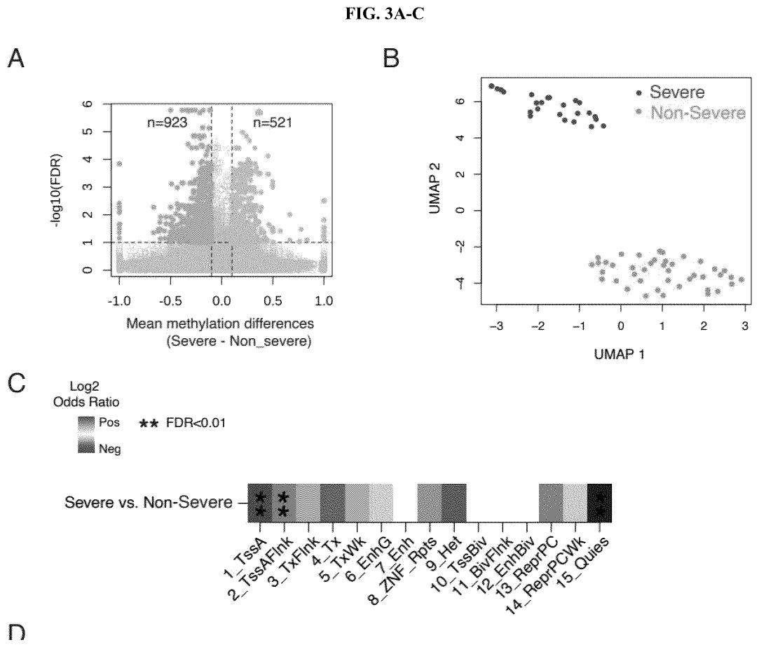

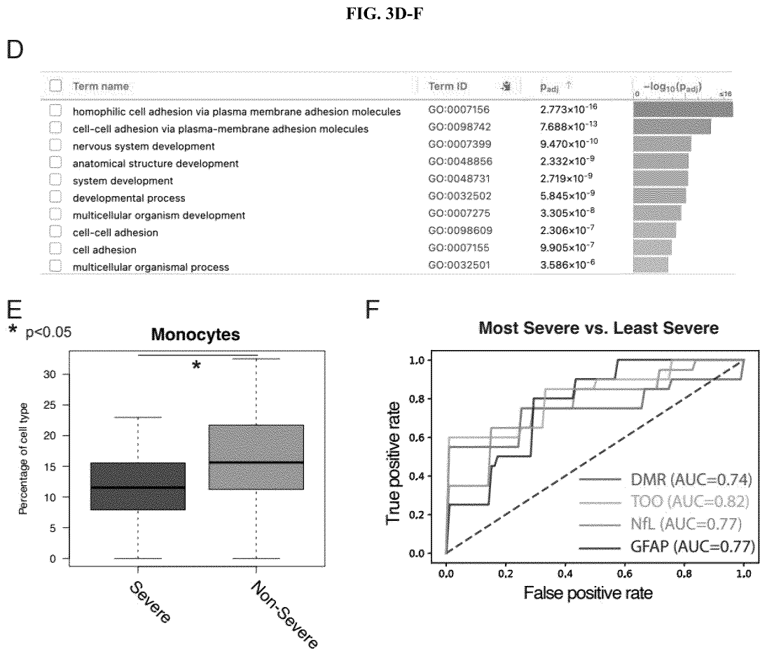

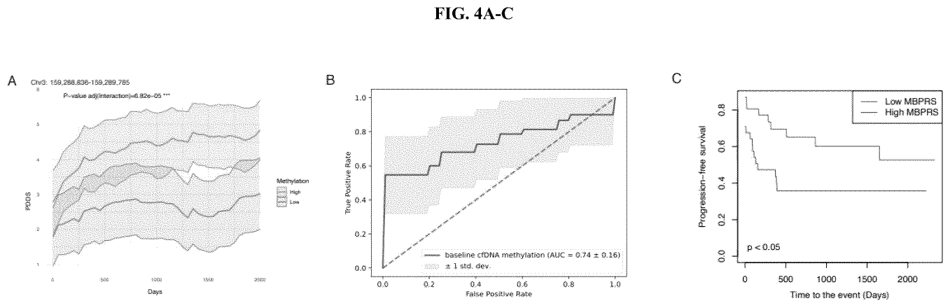

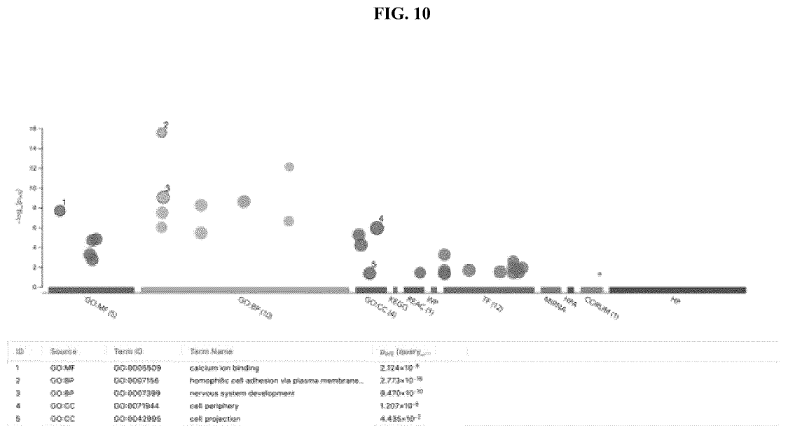

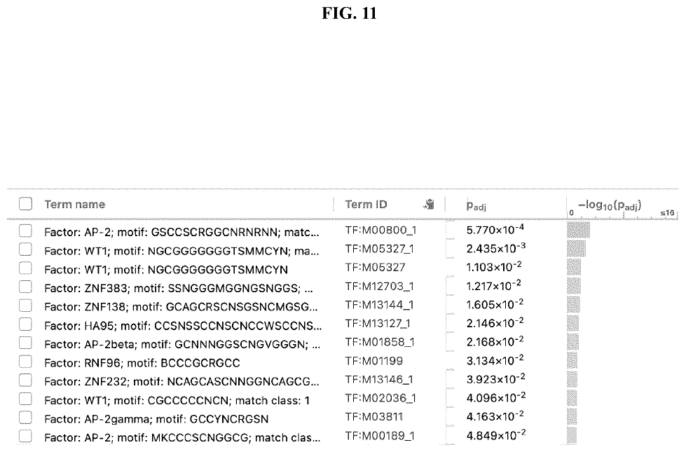

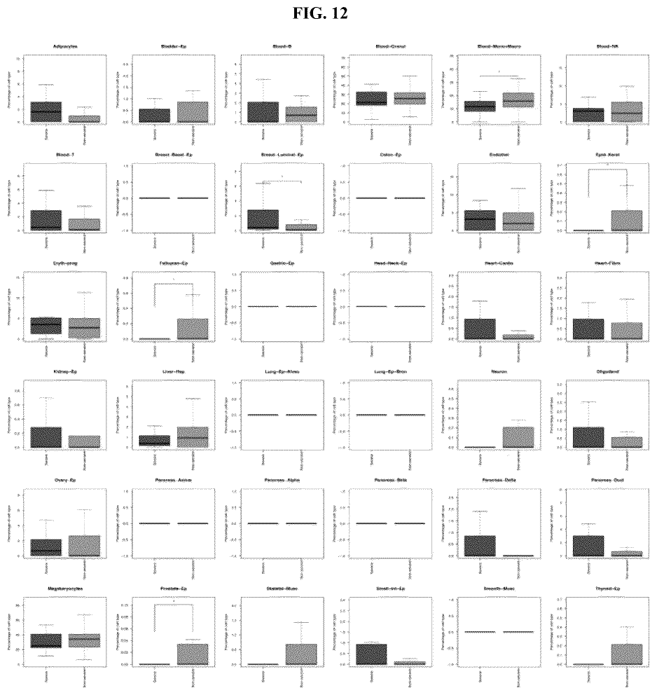

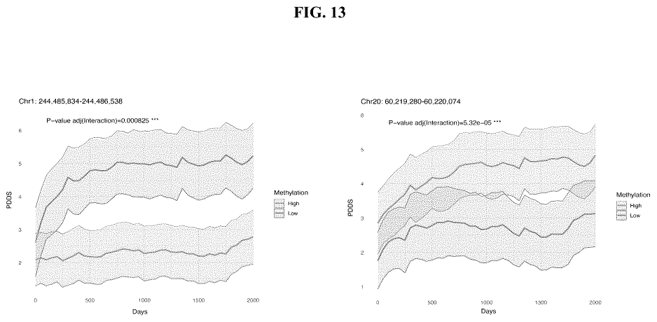

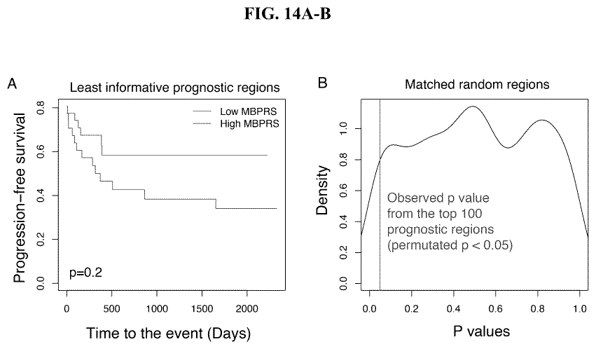

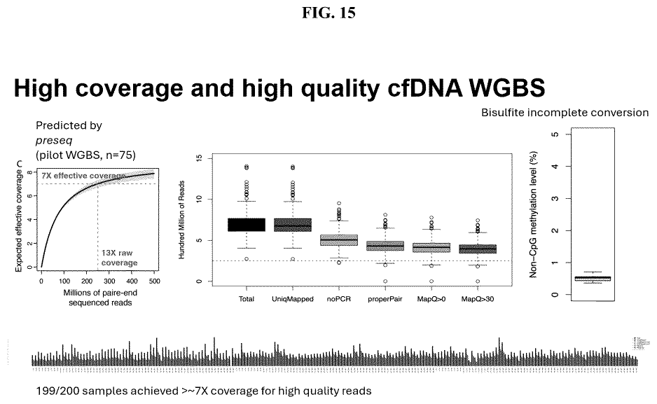

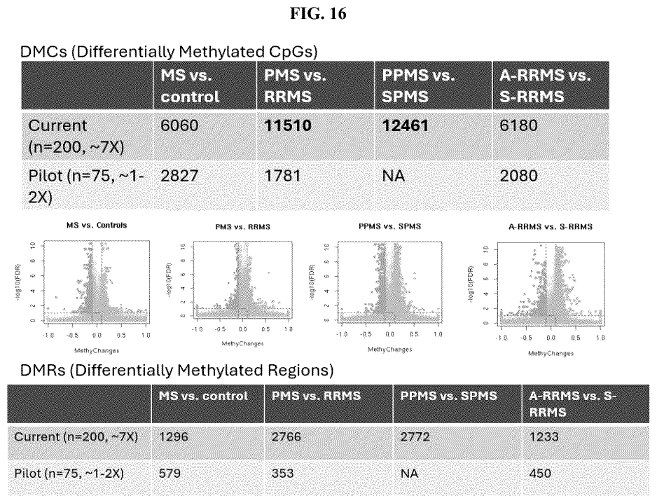

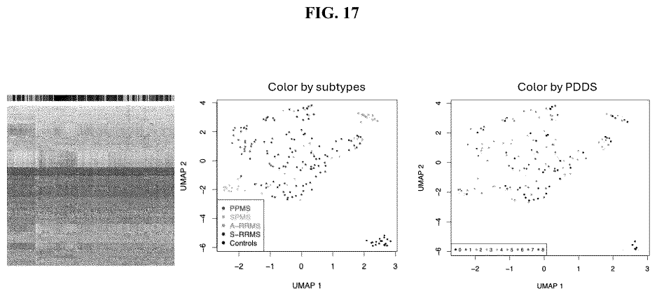

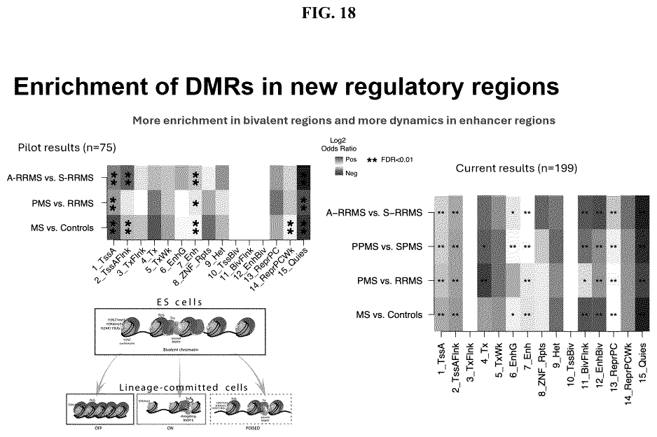

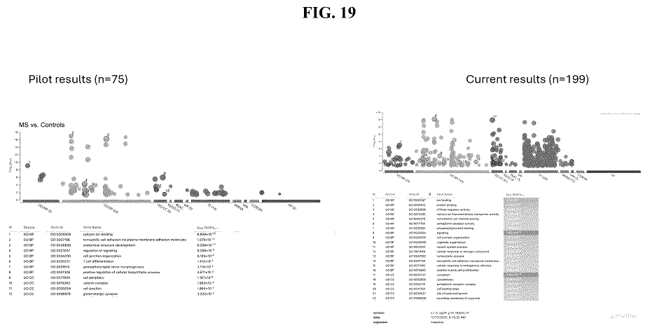

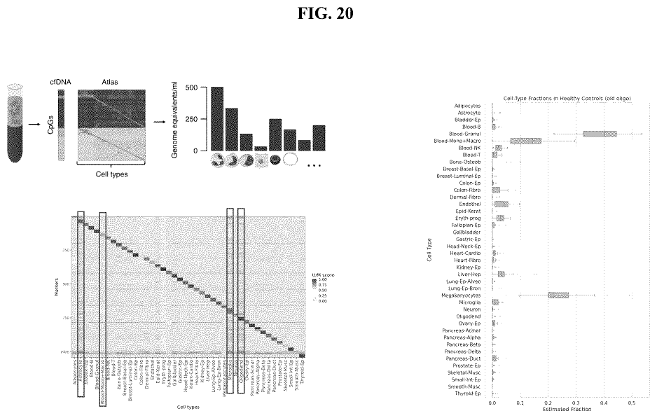

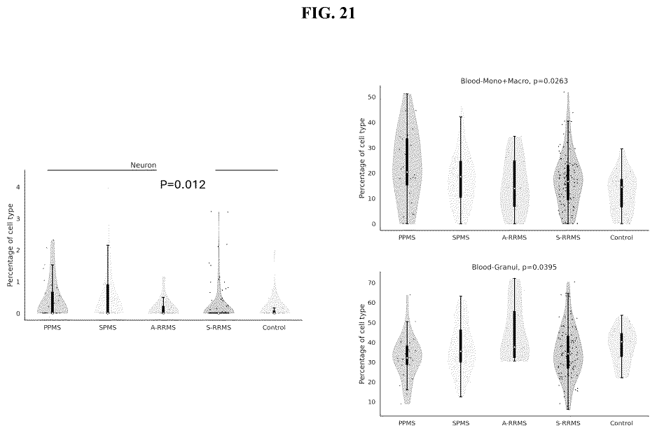

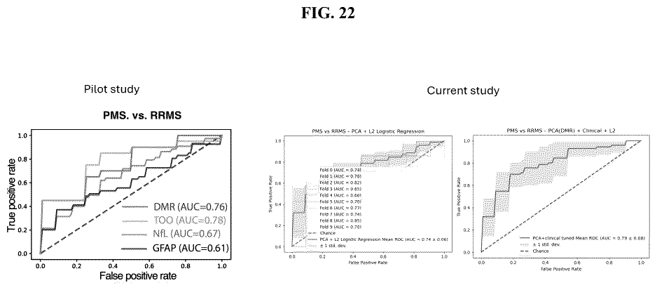

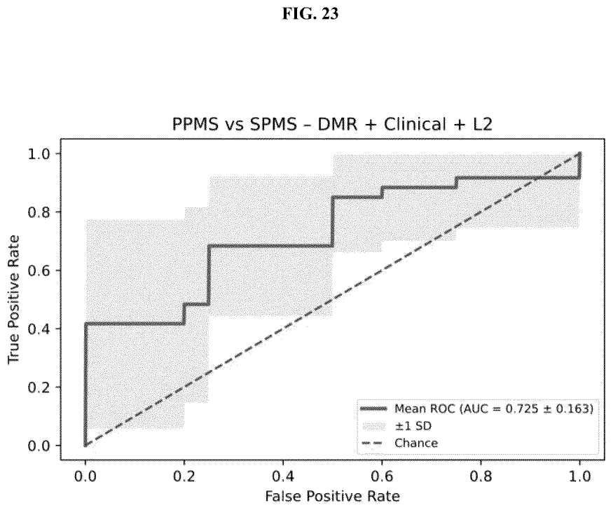

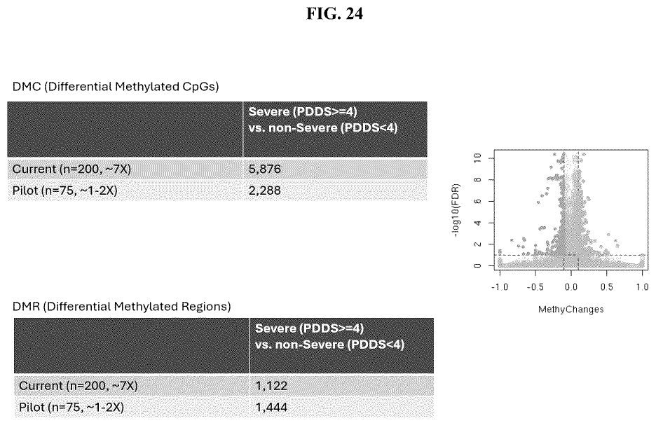

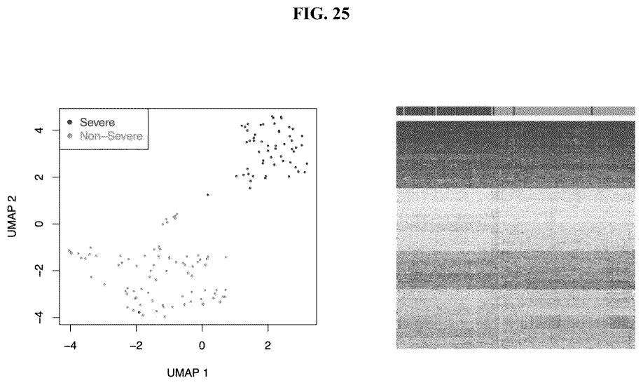

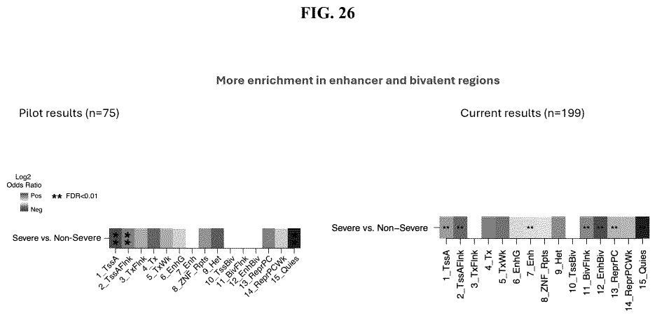

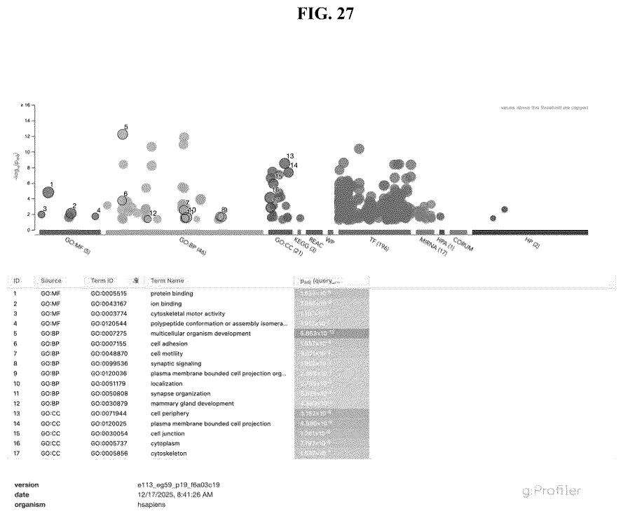

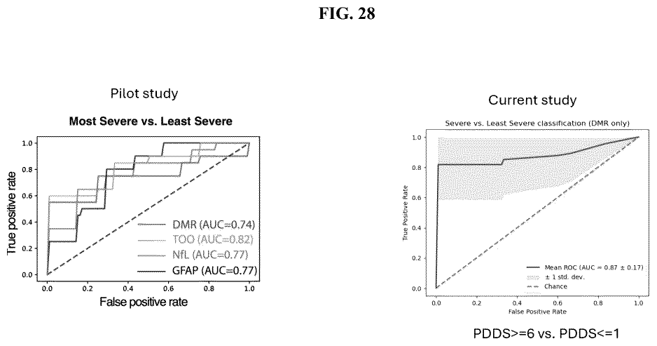

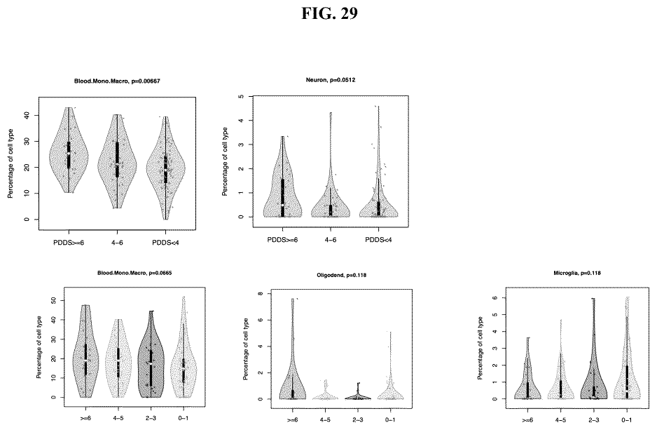

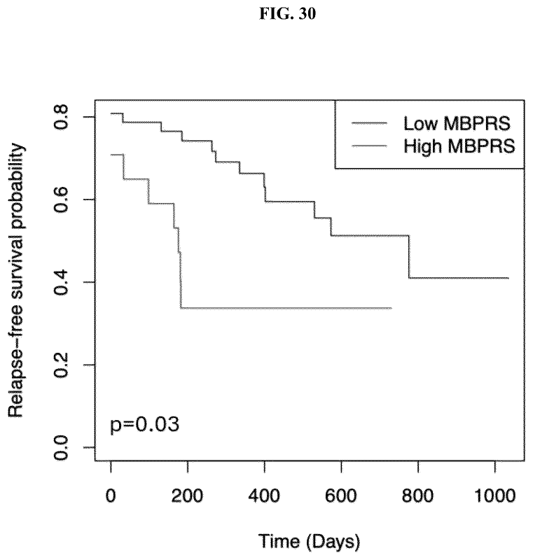

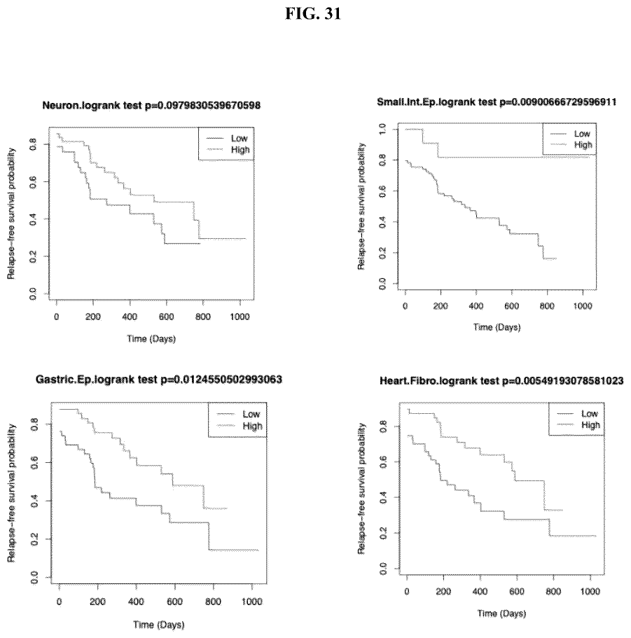

It was evaluated whether cfDNA methylation profiles and tissue-of-origin patterns could distinguish disability severity levels at sample collection (e.g., for disability monitoring). Using a well-validated and already clinically implemented patient-reported outcome (PRO), Patient Determined Disease Steps (PDDS), pwMS were characterized as having severe (PDDS≥4) vs non-severe (normal-mild-moderate, PDDS<4) disability status based on the need for ambulatory assistance around blood collection. (This clinically available PRO is a more convenient outcome than other rater-assessed disability outcomes not collected in current clinical practice.) 521 hypermethylated and 923 hypomethylated CpGs were identified in the severe disability group compared to the non-severe disability group (q value <0.1 and absolute methylation difference >0.1) (FIG. 3A, Supplementary Table 7). DMRs, merged from adjacent differentially methylated CpGs, clearly distinguished these two disability groups (FIG. 3B, FIG. 4B, Supplementary Table 8). Notably, these DMRs were highly enriched in active promoter and TSS-flanking regions and depleted in the quiescent regions (FIG. 3C), indicating strong gene-regulatory potential of these DMRs. Gene Ontology analysis identified significant enrichment of cell adhesion, nervous system development, and anatomical structure development (FIG. 3D, FIG. 10). Motif analysis revealed substantial overrepresentation of multiple transcription factor binding sites, including members of the AP-2 family previously implicated in cranial neural crest cell development52 (FIG. 11; Supplementary Table 9). Tissue-of-origin pattern analysis showed lower monocyte cell death and significant variability in epithelial cell death in the severe disability group (FIG. 3E, FIG. 12; Supplementary Table 10).

The predictive performance of cfDNA methylation profiles in distinguishing pwMS with the most severe disability (PDDS≥6) from those with the least severe (normal-mild) disability (PDDS≤1) was investigated using 10-fold cross-validation. DMRs and differentially inferred tissue-of-origin patterns (in immune and epithelial cells) were identified in each training fold. Then, machine learning models were applied for evaluation in the corresponding held-out test fold. cfDNA methylation DMRs alone achieved AUC 0.74 (SD 0.34), while tissue-of-origin patterns alone yielded AUC 0.82 (SD 0.23) (FIG. 3F). Both approaches performed comparably to historical comparisons with NfL and GFAP (AUC 0.77 each) in a subset of the same cohort, indicating that cfDNA methylation and its tissue-of-origin may predict MS disease severity at blood collection.

### Example 6

cfDNA Methylation Level at Prognostic Regions to Predict Future Disability Progression

It was explored whether baseline cfDNA methylation profiles could predict future disability progression. Using baseline cfDNA methylation levels and longitudinal PDDS scores (Supplementary Table 10), a linear mixed-effects model was fit to assess the interaction between baseline methylation and time. After adjusting for covariates (age, sex, race, DMT type and effectiveness, disease duration, and follow-up time) and correcting for multiple tests, 5,392 “prognostic regions” (FDR<0.01, bonferroni correction) were identified where their baseline methylation levels were significantly associated with the disability severity trajectory over time (FIG. 4A; FIG. 13; Supplementary Table 11). To evaluate the predictive performance, a gradient boosting model was deployed via cross-validation, using baseline methylation at these prognostic regions as features and PDDS scores measured approximately 1,000 days post-blood draw as the outcome. After repeating the procedure 100 times, the model differentiated pwMS with the most severe disability (PDDS≥6) from those with the least (normal-mild) disability (PDDS≤1) (AUC 0.74, SD 0.16; FIG. 4B). A cfDNA methylation-based progression risk score (MBPRS) was computed for each patient using baseline methylation levels at the top 100 (most informative) prognostic regions (see details in Methods). MBPRS stratified patients into significantly different disability progression trajectories (log-rank test p<0.05; FIG. 4C). Two internal control analyses were performed. First, MBPRS using the bottom 100 (least informative) prognostic regions yielded substantially lower discrimination (log-rank test p-0.2; FIG. 14A). Second, MBPRS using the same effect sizes from these top regions but with cfDNA methylation level from 100 randomly selected, matched control regions (matched chromosomes and region sizes) over 1,000 permutations confirmed that the observed separation was highly unlikely to occur by chance (permutation p<0.05, FIG. 14B). Taken together, these findings supported the use of baseline cfDNA methylation level at prognostic regions in forecasting long-term disability progression.

### REFERENCES

The following references are herein incorporated by reference in their entireties.
1. Reich, D. S., Lucchinetti, C. F. & Calabresi, P. A. Multiple Sclerosis. N. Engl. J. Med. 378, 169-180 (2018).
2. Walton, C. et al. Rising prevalence of multiple sclerosis worldwide: Insights from the Atlas of MS, third edition. Mult. Scler. 26, 1816-1821 (2020).
3. Dutta, R. & Trapp, B. D. Relapsing and progressive forms of multiple sclerosis: insights from pathology. Curr. Opin. Neurol. 27, 271-278 (2014).
4. Koch, M., Kingwell, E., Rieckmann, P. & Tremlett, H. The natural history of primary progressive multiple sclerosis. Neurology 73, 1996-2002 (2009).
5. Amin, M. & Hersh, C. M. Updates and advances in multiple sclerosis neurotherapeutics. Neurodegener. Dis. Manag. 13, 47-70 (2023).
6. Robertson, D. & Moreo, N. Disease-modifying therapies in multiple sclerosis: Overview and treatment considerations. Fed. Pract. 33, 28-34 (2016).
7. Pardo, G. & Jones, D. E. The sequence of disease-modifying therapies in relapsing multiple sclerosis: safety and immunologic considerations. J. Neurol. 264, 2351-2374 (2017).
8. Manouchehri, N. et al. Efficacy of disease modifying therapies in progressive MS and how immune senescence may explain their failure. Front. Neurol. 13, 854390 (2022).
9. Hemond, C. C. & Bakshi, R. Magnetic resonance imaging in multiple sclerosis. Cold Spring Harb. Perspect. Med. 8, (2018).
10. Yang, J. et al. Current and Future Biomarkers in Multiple Sclerosis. Int. J. Mol. Sci. 23, (2022).
11. Di Filippo, M. et al. Fluid biomarkers in multiple sclerosis: from current to future applications. Lancet Reg. Health Eur. 44, 101009 (2024).
12. Kuhle, J. et al. Serum neurofilament light chain in early relapsing remitting MS is increased and correlates with CSF levels and with MRI measures of disease severity. Mult. Scler. 22, 1550-1559 (2016).
13. Högel, H. et al. Serum glial fibrillary acidic protein correlates with multiple sclerosis disease severity. Mult. Scler. 26, 210-219 (2020).
14. Ayrignac, X. et al. Serum NfL and GFAP are weak predictors of long-term multiple sclerosis prognosis: A 6-year follow-up. Mult. Scler. Relat. Disord. 89, 105747 (2024).
15. Ferreira-Atuesta, C., Reyes, S., Giovanonni, G. & Gnanapavan, S. The Evolution of Neurofilament Light Chain in Multiple Sclerosis. Front. Neurosci. 15, 642384 (2021).
16. Lei, J. et al. Glial fibrillary acidic protein as a biomarker in severe traumatic brain injury patients: a prospective cohort study. Crit. Care 19, 362 (2015).
17. Cristiano, S. et al. Genome-wide cell-free DNA fragmentation in patients with cancer. Nature 570, 385-389 (2019).
18. Foda, Z. H. et al. Detecting Liver Cancer Using Cell-Free DNA Fragmentomes. Cancer Discov. 13, 616-631 (2023).
19. Mathios, D. et al. Detection and characterization of lung cancer using cell-free DNA fragmentomes. Nat. Commun. 12, 5060 (2021). 20. Liu, Y. At the dawn: cell-free DNA fragmentomics and gene regulation. Br. J. Cancer (2022).
21. Lassmann, H., Brück, W. & Lucchinetti, C. F. The immunopathology of multiple sclerosis: an overview. Brain Pathol. 17, 210-218 (2007).
22. Lo, Y. M. D., Han, D. S. C., Jiang, P. & Chiu, R. W. K. Epigenetics, fragmentomics, and topology of cell-free DNA in liquid biopsies. Science 372, (2021).
23. Liggett, T. et al. Methylation patterns of cell-free plasma DNA in relapsing-remitting multiple sclerosis. J. Neurol. Sci. 290, 16-21 (2010).
24. Lehmann-Werman, R. et al. Identification of tissue-specific cell death using methylation patterns of circulating DNA. Proc. Natl. Acad. Sci. U.S.A. 113, E1826-34 (2016).
25. Dunaeva, M., Derksen, M. & Pruijn, G. J. M. LINE-1 Hypermethylation in Serum Cell-Free DNA of Relapsing Remitting Multiple Sclerosis Patients. Mol. Neurobiol. 55, 4681-4688 (2018).
26. Celarain, N. & Tomas-Roig, J. Aberrant DNA methylation profile exacerbates inflammation and neurodegeneration in multiple sclerosis patients. J. Neuroinflammation 17, 21 (2020).
27. Chomyk, A. M. et al. DNA methylation in demyelinated multiple sclerosis hippocampus. Sci. Rep. 7, 8696 (2017).
28. Maltby, V. E. et al. Genome-wide DNA methylation profiling of CD8+ T cells shows a distinct epigenetic signature to CD4+ T cells in multiple sclerosis patients. Clin. Epigenetics 7, 118 (2015).
29. Graves, M. C. et al. Methylation differences at the HLA-DRB1 locus in CD4+ T-Cells are associated with multiple sclerosis. Mult. Scler. 20, 1033-1041 (2014).
30. Kulakova, O. G. et al. Whole-genome DNA methylation analysis of peripheral blood mononuclear cells in multiple sclerosis patients with different disease courses. Acta Naturae 8, 103-110 (2016).
31. Huynh, J. L. et al. Epigenome-wide differences in pathology-free regions of multiple sclerosis-affected brains. Nat. Neurosci. 17, 121-130 (2014).
32. Bos, S. D. et al. Genome-wide DNA methylation profiles indicate CD8+ T cell hypermethylation in multiple sclerosis. PLOS One 10, e0117403 (2015).
33. Moss, J. et al. Comprehensive human cell-type methylation atlas reveals origins of circulating cell-free DNA in health and disease. Nat. Commun. 9, 5068 (2018).
34. Liu, Y. et al. FinaleMe: Predicting DNA methylation by the fragmentation patterns of plasma cell-free DNA. Nat. Commun. 15, 2790 (2024).
35. Loyfer, N. et al. A DNA methylation atlas of normal human cell types. Nature 613, 355-364 (2023).
36. Li, S. et al. Comprehensive tissue deconvolution of cell-free DNA by deep learning for disease diagnosis and monitoring. Proc. Natl. Acad. Sci. U.S.A. 120, e2305236120 (2023).
37. Sun, K. et al. Plasma DNA tissue mapping by genome-wide methylation sequencing for noninvasive prenatal, cancer, and transplantation assessments. Proc. Natl. Acad. Sci. U.S.A. 112, E5503-12 (2015).
38. Dolzhenko, E. & Smith, A. D. Using beta-binomial regression for high-precision differential methylation analysis in multifactor whole-genome bisulfite sequencing experiments. BMC Bioinformatics 15, 215 (2014).
39. Nichols, R. V. et al. High-throughput robust single-cell DNA methylation profiling with sciMETv2. Nat. Commun. 13, 7627 (2022).
40. Fang, R. et al. Comprehensive analysis of single cell ATAC-seq data with SnapATAC. Nat. Commun. 12, 1337 (2021).
41. Klema, V. & Laub, A. The singular value decomposition: Its computation and some applications. IEEE Trans. Automat. Contr. 25, 164-176 (1980).
42. McInnes, L., Healy, J., Saul, N. & Großberger, L. UMAP: Uniform Manifold Approximation and Projection. J. Open Source Softw. 3, 861 (2018).
43. Roadmap Epigenomics Consortium et al. Integrative analysis of 111 reference human epigenomes. Nature 518, 317-330 (2015).
44. Ernst, J. & Kellis, M. ChromHMM: automating chromatin-state discovery and characterization. Nat. Methods 9, 215-216 (2012).
45. Deng, Y. et al. Zbtb 14 regulates monocyte and macrophage development through inhibiting pu.1 expression in zebrafish. Elife 11, (2022).
46. Legnini, I. et al. Circ-ZNF609 is a circular RNA that can be translated and functions in myogenesis. Mol. Cell 66, 22-37.e9 (2017).
47. Gauthier, M. et al. A defective Krab-domain zinc-finger transcription factor contributes to altered myogenesis in myotonic dystrophy type 1. Hum. Mol. Genet. 22, 5188-5198 (2013).
48. Kaskow, B. J. & Baecher-Allan, C. Effector T cells in multiple sclerosis. Cold Spring Harb. Perspect. Med. 8, (2018).
49. Ajami, B., Bennett, J. L., Krieger, C., McNagny, K. M. & Rossi, F. M. V. Infiltrating monocytes trigger EAE progression, but do not contribute to the resident microglia pool. Nat. Neurosci. 14, 1142-1149 (2011).
50. Dziedzic, A., Miller, E., Bijak, M., Przyslo, L. & Saluk-Bijak, J. Increased pro-thrombotic platelet activity associated with thrombin/PAR1-dependent pathway disorder in patients with secondary progressive multiple sclerosis. Int. J. Mol. Sci. 21, 7722 (2020).
51. Zhu, W. et al. Association between serum multi-protein biomarker profile and real-world disability in multiple sclerosis. Brain Commun. 6, fcad300 (2024).
52. Eckert, D., Buhl, S., Weber, S., Jäger, R. & Schorle, H. The AP-2 family of transcription factors. Genome Biol. 6, 246 (2005).
53. Akaishi, T., Takahashi, T. & Nakashima, I. Peripheral blood monocyte count at onset may affect the prognosis in multiple sclerosis. J. Neuroimmunol. 319, 37-40 (2018).
54. Peng, H.-R. et al. Intestinal epithelial dopamine receptor signaling drives sex-specific disease exacerbation in a mouse model of multiple sclerosis. Immunity 56, 2773-2789.e8 (2023).
55. Krueger, F. Trim galore. A wrapper tool around Cutadapt and FastQC to consistently apply quality and adapter trimming to FastQ files 516, 517 (2015).
56. Martin, M. Cutadapt removes adapter sequences from high-throughput sequencing reads. EMBnet J. 17, 10 (2011).
57. Zhou, W. et al. BISCUIT: an efficient, standards-compliant tool suite for simultaneous genetic and epigenetic inference in bulk and single-cell studies. Nucleic Acids Res. 52, e32 (2024).
58. Faust, G. G. & Hall, I. M. SAMBLASTER: fast duplicate marking and structural variant read extraction. Bioinformatics 30, 2503-2505 (2014).
59. Liu, Y., Siegmund, K. D., Laird, P. W. & Berman, B. P. Bis-SNP: combined DNA methylation and SNP calling for Bisulfite-seq data. Genome Biol. 13, R61 (2012).
60. Perez, G. et al. The UCSC Genome Browser database: 2025 update. Nucleic Acids Res. 53, D1243-D1249 (2025).
61. Kolberg, L. et al. g: Profiler-interoperable web service for functional enrichment analysis and gene identifier mapping (2023 update). Nucleic Acids Res. 51, W207-W212 (2023).
62. Bates, D., Mächler, M., Bolker, B. & Walker, S. Fitting linear mixed-effects models Usinglme4. J. Stat. Softw. 67, (2015).
63. Kuznetsova, A., Brockhoff, P. B. & Christensen, R. H. B. LmerTest package: Tests in linear mixed effects models. J. Stat. Softw. 82, (2017).
64. Virtanen, P. et al. SciPy 1.0: fundamental algorithms for scientific computing in Python. Nat. Methods 17, 261-272 (2020).
65. Quinlan, A. R. & Hall, I. M. BEDTools: a flexible suite of utilities for comparing genomic features. Bioinformatics 26, 841-842 (2010).
66. Buitinck, L. et al. API design for machine learning software: experiences from the scikit-learn project. arXiv [cs.LG] (2013).
67. Chen, T. & Guestrin, C. XGBoost: A Scalable Tree Boosting System. arXiv [cs.LG] (2016).

1. A method comprising detecting the methylation status of a plurality of biomarkers in a sample comprising cell free DNA (cfDNA) from a subject, wherein each of the plurality of biomarkers is a methylation locus comprising at least a single CpG site and/or differentially methylated region (DMR) that is indicative of multiple sclerosis (MS) status.

2. The method of claim 1, wherein the one or more of the plurality of biomarkers is indicative of a subject suffering from MS versus not suffering from MS.

3. The method of claim 1, wherein the one or more of the plurality of biomarkers is indicative of a subject suffering from progressive MS (PMS) versus suffering from relapsing-remitting MS (RRMS).

4. The method of claim 1, wherein the one or more of the plurality of biomarkers is indicative of a subject suffering from RRMS versus suffering from PMS.

5. The method of claim 1, wherein the one or more of the plurality of biomarkers is indicative of a subject suffering from primary progressive MS (PPMS) versus suffering from secondary progressive MS (SPMS).

6. The method of claim 1, wherein the one or more of the plurality of biomarkers is indicative of a subject suffering from SPMS versus suffering from PPMS.

7. The method of claim 1, wherein the one or more of the plurality of biomarkers is indicative of a subject suffering severe MS (PDDS≥4) versus suffering from non-severe MS (PDDS<4).

8. The method of claim 1, wherein the one or more of the plurality of biomarkers is indicative of a subject suffering non-severe MS (PDDS<4) versus suffering from severe MS (PDDS≥4).

9. The method of claim 1, wherein the one or more of the plurality of biomarkers is indicative of a subject in active relapse of MS versus stable remission from MS.

10. The method of claim 1, wherein the one or more of the plurality of biomarkers is indicative of a subject in stable remission from MS versus active relapse of MS.

11. The method of claim 1, wherein the plurality of biomarkers are selected from the CpGs of Supplementary Table 3.

12. The method of claim 1, wherein the plurality of biomarkers are selected from the DMRs of Supplementary Table 4.

13. The method of claim 1, wherein the plurality of biomarkers are selected from the CpGs of Supplementary Table 7.

14. The method of claim 1, wherein the plurality of biomarkers are selected from the DMRs of Supplementary Table 8.

15. A method of treating a subject comprising conducting the method of claim 1, and administering to the subject a treatment for MS.

16. The method of claim 1, wherein the plurality of biomarkers comprises 20 or more biomarkers.

17. The method of claim 1, wherein the plurality of biomarkers comprises 2000 or fewer biomarkers.

18. A panel of biomarkers wherein each of the biomarkers is a methylation locus comprising at least a single CpG site and/or differentially methylated region (DMR) that is indicative of multiple sclerosis (MS) status.

19-31. (canceled)

32. A kit comprising reagents for conducting the method of claim 1.
